(19) United States
(12) Patent Application Publication
(10) Pub. No.: US 2026/0183270 A1
(43) Pub. Date: Jul. 2, 2026

(54) PHARMACEUTICAL COMPOSITION COMPRISING AAK1 INHIBITOR

(71) Applicant: Xizang Haisco Pharmaceutical Co., Ltd., Lhoka, Tibet (CN)

(72) Inventors: Xuanmiao ZHANG, Lhoka, Tibet (CN); Hua MAO, Lhoka, Tibet (CN); Hang SONG, Lhoka, Tibet (CN); Shihao DENG, Lhoka, Tibet (CN); Yang ZHOU, Lhoka, Tibet (CN); Ying DOU, Lhoka, Tibet (CN)

(21) Appl. No.: 19/129,194

(22) PCT Filed: Nov. 10, 2023
(86) PCT No.: PCT/CN2023/130976

§ 371 Date: May 12, 2025

(30) Foreign Application Priority Data
Nov. 10, 2022 (CN) 202211406281.1

(51) Int. Cl. A61K 31/4433; A61K 47/02; A61K 47/36; A61P 25/04; C07D 401/04

(52) U.S. Cl. CPC A61K 31/4433; A61K 47/02; A61K 47/36; A61P 25/04; C07D 401/04

(57) ABSTRACT

Provided are a pharmaceutical composition of a compound of formula (I) or a stereoisomer or a pharmaceutically acceptable salt thereof, and a pharmaceutical formulation thereof; and a preparation method for the pharmaceutical composition and the pharmaceutical formulation, and use of the pharmaceutical composition and the pharmaceutical formulation in the preparation of a medicament for treating an AAK1-induced related disease by inhibition or degradation of AAK1.

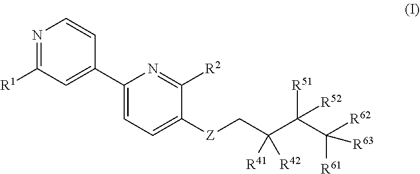

### CROSS-REFERENCE TO RELATED APPLICATION

This application is a 35 U.S.C. § 371 National Stage of International Patent Application No. PCT/CN2023/130976, filed Nov. 10, 2023, designating the United States, which claims priority to and the benefits of Chinese Patent Application No. 202211406281.1, filed Nov. 10, 2022, the disclosures of which are incorporated herein in their entirety by reference, and priority is claimed to each of the foregoing.

### TECHNICAL FIELD

The present disclosure relates to a pharmaceutical composition of a compound of formula (I) or a stereoisomer or a pharmaceutically acceptable salt thereof, and a pharmaceutical formulation thereof, and the use of the pharmaceutical composition and the pharmaceutical formulation in the preparation of a medicament for treating an AAK1-induced related disease by inhibition of AAK1.

### BACKGROUND

Neuropathic pain (NP) is a general term for a range of pains caused by damage and diseases of the somatic sensory nervous system. It is divided into peripherally-induced neuropathic pain (pNP) and central neuropathic pain. pNP is more common clinically, and can be divided into diabetic peripheral neuralgia, postherpetic neuralgia, trigeminal neuralgia, postoperative chronic neuralgia, etc. Clinically, pNP patients often have symptoms such as spontaneous pain (pain without any external stimulation), allodynia (increased response to painful stimuli), hyperalgesia (feeling pain in response to stimuli that are normally painless) and paresthesia, which seriously affect the patients' quality of life.

AAK1 is a member of the Ark1/Prk1 family of serine/threonine kinases and is widely expressed in the brain and spinal cord. Studies have shown that AAK1 knockout mice exhibit responses to persistent pain or hypoalgesia, and AAK1 knockout mice do not develop hyperalgesia in the neuropathic pain model of spinal nerve ligation, which confirms that AAK1 is a feasible target for the treatment of pNP.

We provide an AAK1 inhibitor, but the compound has a low melting point and often adheres to the capsule punch or tablet punch when being prepared into tablets or capsules. Based on this, we intend to provide a composition containing an AAK1 inhibitor, which can be advantageously applied to the preparation of conventional solid formulations, and the resulting formulation has good in vitro dissolution behavior and stability.

### SUMMARY OF THE INVENTION

The present disclosure provides a pharmaceutical composition of a compound of formula (I) and a stereoisomer and a pharmaceutically acceptable salt thereof, and a pharmaceutical formulation thereof.

The pharmaceutical composition or the pharmaceutical formulation of the present disclosure has good solubility, dissolution rate, bioavailability, oral performance, stable quality, good safety and low irritation, and meets the drug quality standards.

In one aspect, the present disclosure provides a pharmaceutical composition, comprising:
an active ingredient, wherein the active ingredient is selected from a compound of formula (I), or a stereoisomer or a pharmaceutically acceptable salt thereof:

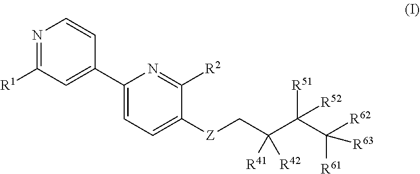

wherein
Z is selected from NH or O;
R¹ and R² are each independently selected from H, deuterium, halogen, amino, —COOH, cyano, sulfonyl, aminoacyl, C₁₋₆ alkyl, halo C₁₋₆ alkyl, or deuterated C₁₋₆ alkyl, wherein the alkyl is optionally further substituted with 1-3 R^{A} substituents;
R⁴¹ and R⁴² are each independently selected from H, deuterium, amino, C₁₋₆ alkyl, halogen, cyano, hydroxyl, halo C₁₋₆ alkyl, or deuterated C₁₋₆ alkyl;
R⁵¹ and R⁵² are each independently selected from H, deuterium, amino, or halogen;
R⁶¹, R⁶² and R⁶³ are each independently selected from H, deuterium, halogen, amino, cyano, hydroxyl, C₁₋₆ alkyl, halo C₁₋₆ alkyl, or deuterated C₁₋₆ alkyl;
or, R⁵¹ and R⁶¹, or R⁶¹ and R⁶² together with the carbon atom(s) to which they are each attached form a double bond;
R^{A} is selected from deuterium, halogen, amino, cyano, hydroxyl, C₁₋₆ alkyl, halo C₁₋₆ alkyl, deuterated C₁₋₆ alkyl, C₁₋₆ alkoxy, halo C₁₋₆ alkoxy, deuterated C₁₋₆ alkoxy, or hydroxy C₁₋₆ alkyl,
provided that when Z is selected from O,

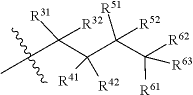

does not form the following structures:

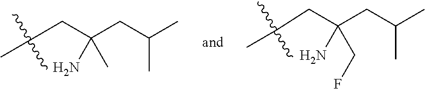

a non-active ingredient;
wherein the active ingredient is present in the pharmaceutical composition in an amount of 5% to 90% w/w, preferably 5% to 80% w/w, and more preferably 10% to 80% w/w;
in some embodiments, the active ingredient (calculated as a free base) is present in the pharmaceutical composition in an amount of 5% to 85% w/w, preferably 8% to 80% w/w, and more preferably 10% to 80% w/w;
in some embodiments, the active ingredient (calculated as a free base) is present in the pharmaceutical composition in an amount of 8% to 85% w/w, preferably 10% to 85% w/w, and more preferably 10% to 80% w/w;
in some embodiments, the active ingredient (calculated as a free base) is present in the pharmaceutical composition in an amount of 5% to 80% w/w, 5% to 70% w/w, 5% to 60% w/w, 5% to 50% w/w, 5% to 40% w/w, 5% to 30% w/w, 5% to 20% w/w, 10% to 80% w/w, 10% to 70% w/w, 10% to 60% w/w, 10% to 50% w/w, 10% to 40% w/w, 10% to 30% w/w, or 10% to 20% w/w.

In some embodiments, in the compound of formula (I), Z is O;
R¹ and R² are selected from halo C₁₋₂ alkyl;
R⁵¹ and R⁵² are each independently selected from H or deuterium;
R⁴¹ and R⁴² are each independently selected from amino or C₁₋₂ alkyl;
R⁶¹, R⁶² and R⁶³ are each independently selected from H, deuterium, C₁₋₂ alkyl, or halo C₁₋₂ alkyl;
or, R⁵¹ and R⁶¹, or R⁶¹ and R⁶² together with the carbon atom(s) to which they are each attached form a double bond;
in some embodiments, in the compound of formula (I),
R¹ and R² are each independently selected from —CH₂F, —CHF₂, —CF₃, —CH₂CH₂F, —CH₂CHF₂, —CH₂CF₃, —CHFCH₂F, —CHFCHF₂, —CHFCF₃, —CF₂CH₂F, —CF₂CHF₂, —CF₂CF₃, —CH₂Cl, —CHCl₂, —CCl₃, —CH₂CH₂Cl, —CH₂CHCl₂, —CH₂CCl₃, —CHClCH₂Cl, —CHClCHCl₂, —CHClCCl₃, —CCl₂CH₂Cl, —CCl₂CHCl₂, or —CCl₂CCl₃;
R⁵¹ and R⁵² are each independently selected from H or deuterium;
R⁴¹ and R⁴² are each independently selected from amino, —CH₃, or —CH₂CH₃;
R⁶¹, R⁶² and R⁶³ are each independently selected from H, deuterium, —CH₃, —CH₂CH₃, —CH₂F, —CHF₂, —CF₃, —CH₂CH₂F, —CH₂CHF₂, —CH₂CF₃, —CHFCH₂F, —CHFCHF₂, —CHFCF₃, —CF₂CH₂F, —CF₂CHF₂, —CF₂CF₃, —CH₂Cl, —CHCl₂, —CCl₃, —CH₂CH₂Cl, —CH₂CHCl₂, —CH₂CCl₃, —CHClCH₂Cl, —CHClCHCl₂, —CHClCCl₃, —CCl₂CH₂Cl, —CCl₂CHCl₂, or —CCl₂CCl₃;
or, R⁵¹ and R⁶¹, or R⁶¹ and R⁶² together with the carbon atom(s) to which they are each attached form a double bond;
in some embodiments, in the compound of formula (I),
Z is O;
R¹ and R² are each independently selected from —CH₂F, —CHF₂, or —CF₃;
R⁵¹ and R⁵² are each independently selected from H or deuterium;
R⁴¹ and R⁴² are each independently selected from amino or —CH₃;
R⁶¹, R⁶² and R⁶³ are each independently selected from H, deuterium, —CH₃, or CF₃;
in some embodiments, in the compound of formula (I),
Z is O;
R¹ and R² are each independently selected from —CH₂F, —CHF₂, or —CF₃;
R⁵¹ and R⁵² together with the carbon atom(s) to which they are each attached form a double bond;
R⁴¹ and R⁴² are each independently selected from amino or —CH₃;
R⁶¹, R⁶² and R⁶³ are each independently selected from H, deuterium, —CH₃, or CF₃;
in some embodiments, in the compound of formula (I),
Z is O;
R¹ and R² are each independently selected from —CH₂F, —CHF₂, or —CF₃;
R⁵¹ and R⁵² are each independently selected from H or deuterium;
R⁴¹ and R⁴² are each independently selected from amino or —CH₃; each R⁶³ is independently selected from H, deuterium, —CH₃, or CF₃;
R⁶¹ and R⁶² together with the carbon atom(s) to which they are each attached form a double bond;
in some embodiments, the compound of formula (I) is selected from one of the following structures:

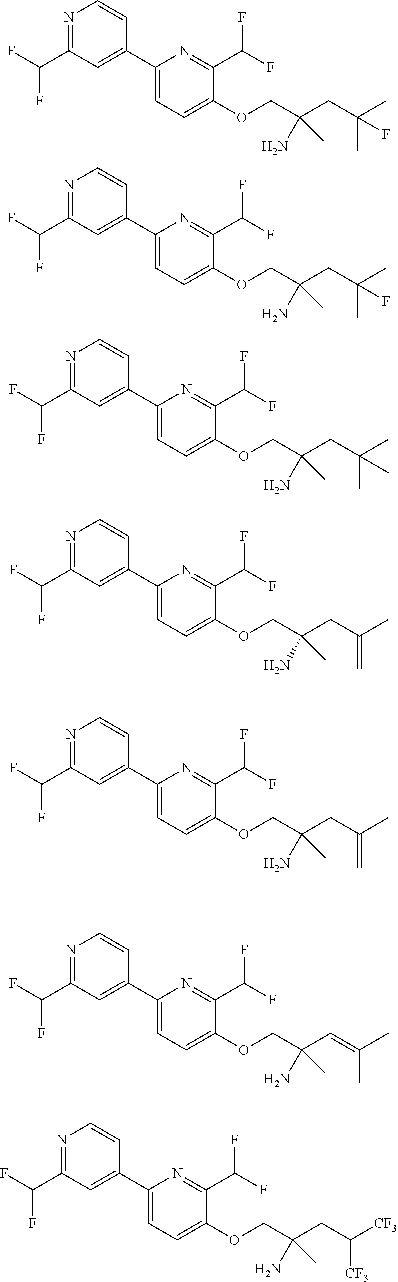

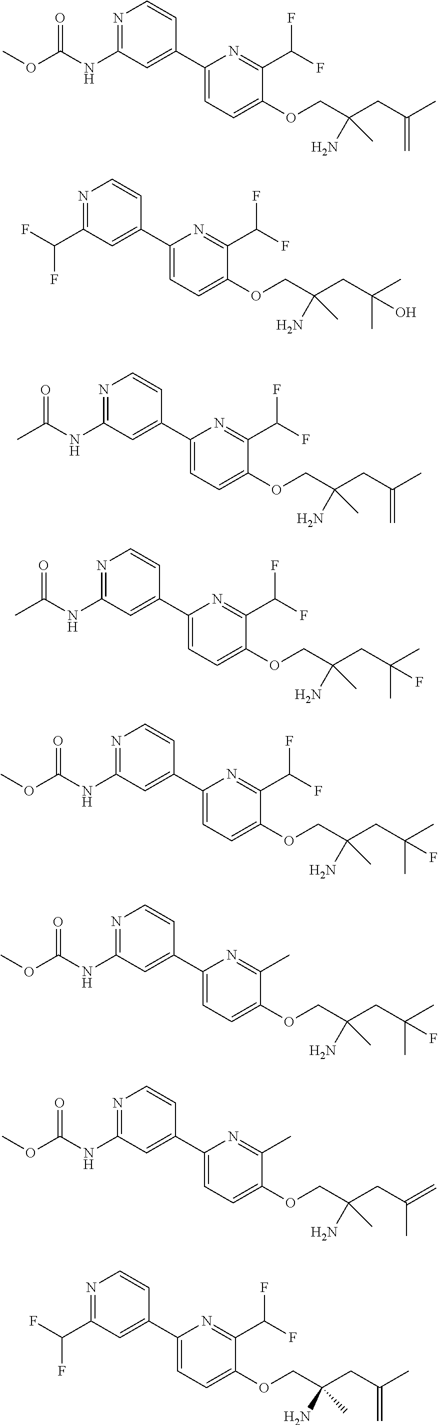

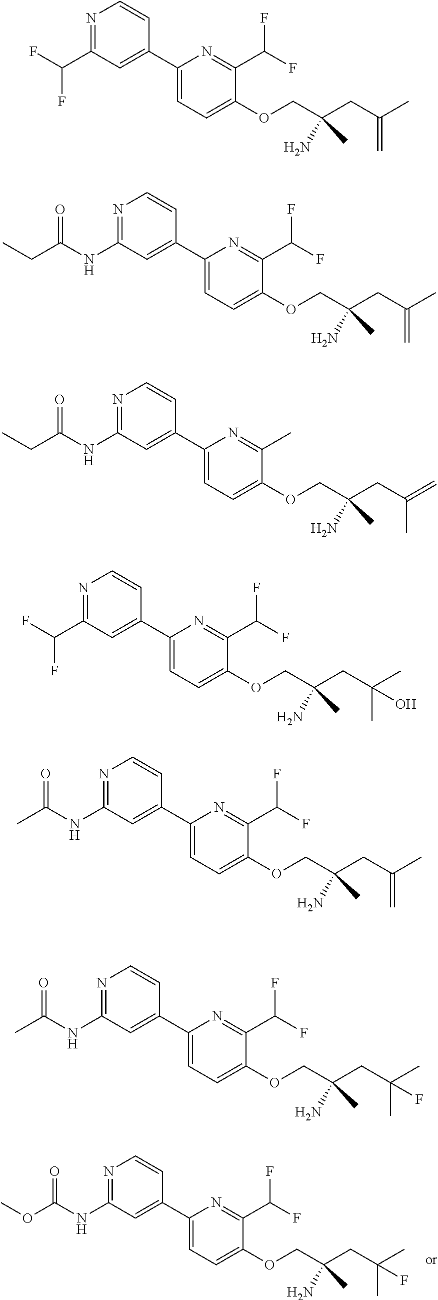

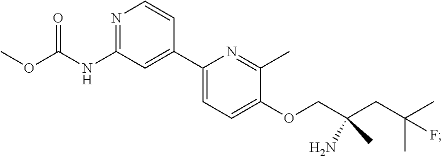 [SMILES: COC(=O)Nc1cc(-c2ccc(OC[C@@](C)(N)CC(C)(C)F)c(C)n2)ccn1]

in some embodiments, the compound of formula (I) is selected from one of the following structures:

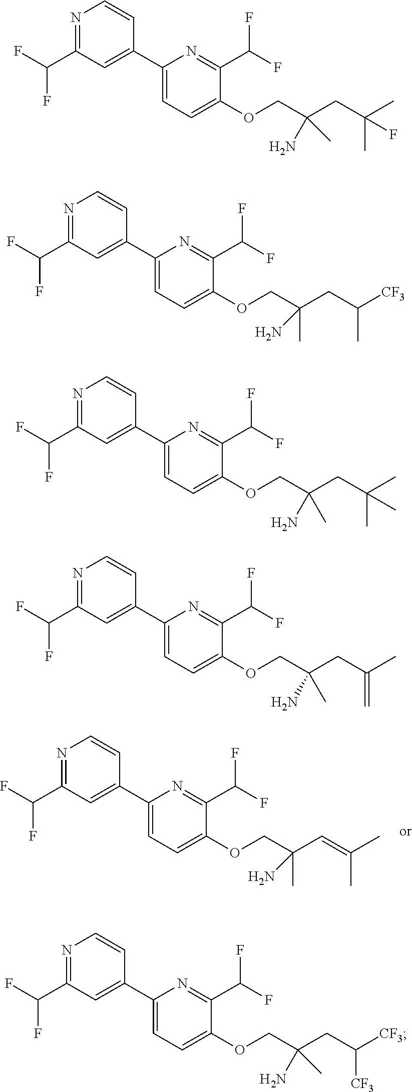 [SMILES: CC(C)(F)CC(C)(N)COc1ccc(-c2ccnc(C(F)F)c2)nc1C(F)F ; CC(CC(C)(N)COc1ccc(-c2ccnc(C(F)F)c2)nc1C(F)F)C(F)(F)F ; CC(C)(C)CC(C)(N)COc1ccc(-c2ccnc(C(F)F)c2)nc1C(F)F ; C=C(C)C[C@](C)(N)COc1ccc(-c2ccnc(C(F)F)c2)nc1C(F)F ; CC(C)=CC(C)(N)COc1ccc(-c2ccnc(C(F)F)c2)nc1C(F)F ; CC(N)(COc1ccc(-c2ccnc(C(F)F)c2)nc1C(F)F)CC(C(F)(F)F)C(F)(F)F]

in some embodiments, the non-active ingredient contains an infiltrating agent;
in some embodiments, the non-active ingredient contains a disintegrant;
in some embodiments, the non-active ingredient contains a diluent;
in some embodiments, the non-active ingredient contains a lubricant;
in some embodiments, the infiltrating agent is selected from one or more of silicates;
preferably, the infiltrating agent is one or more of silica, magnesium silicate, magnesium trisilicate, magnesium aluminum silicate and talc; more preferably, the infiltrating agent is one or more of fumed silica, precipitated silica, sol-gel silica, magnesium silicate, magnesium trisilicate, magnesium aluminum silicate and talc;
in some embodiments, the disintegrant is selected from one or more of croscarmellose sodium, crospovidone, starch and a derivative thereof, low-substituted hydroxypropyl cellulose, low-substituted sodium hydroxymethyl cellulose, a surfactant, alginic acid and sodium alginate, and clays; preferably, the disintegrant is one or more of croscarmellose sodium, low-substituted hydroxypropyl cellulose, low-substituted sodium hydroxymethyl cellulose, crospovidone, starch, sodium carboxymethyl starch, hydroxypropyl starch, polysorbate 80, sodium lauryl sulfate, bentonite, and colloidal magnesium aluminum silicate; more preferably, the disintegrant is one or more of croscarmellose sodium, low-substituted hydroxypropyl cellulose, low-substituted sodium hydroxymethyl cellulose, sodium carboxymethyl starch, and crospovidone;
in some embodiments, the diluent is selected from one or more of starch, pregelatinized starch, dextrin, lactose monohydrate, anhydrous lactose, sucrose, microcrystalline cellulose, inorganic salts, and sugar alcohols; preferably, the diluent is one or more of pregelatinized starch, dextrin, lactose, sucrose, microcrystalline cellulose, calcium sulfate, calcium hydrogenphosphate dihydrate, anhydrous calcium hydrogenphosphate, calcium phosphate, calcium carbonate, calcium stearate, magnesium oxide, aluminum hydroxide, mannitol, xylitol, and sorbitol; more preferably, the diluent is one or more of pregelatinized starch, lactose, sucrose, microcrystalline cellulose, calcium hydrogenphosphate dihydrate, anhydrous calcium hydrogenphosphate, calcium phosphate, mannitol, and dextrin;
in some embodiments, the lubricant is selected from one or more of talc, stearic acid, metal stearate, stearate, glyceryl behenate, sodium lauryl sulfate or colloidal silica; in some embodiments, the lubricant is selected from one or more of talc, stearic acid, metal stearate, stearate, glyceryl behenate, and sodium lauryl sulfate; preferably, the lubricant is one or more of talc, calcium stearate, magnesium stearate and zinc stearate, polyoxyethylene stearate, glyceryl monostearate, and glyceryl palmitostearate; more preferably, the lubricant is one or more of talc, calcium stearate, magnesium stearate, and glyceryl monostearate; more preferably, the lubricant is one or more of magnesium stearate, talc or colloidal silica;
in some embodiments, the weight ratio of the active ingredient to the infiltrating agent is 1:0.05 to 1:5, preferably 1:0.08 to 1:3, further preferably 1:0.12 to 1:2, and more preferably 1:0.125, 1:0.2, 1:0.25, 1:0.375, 1:0.5, 1:0.5, 1:1, 1:1.5, or 1:2;
in some embodiments, the weight ratio of the active ingredient to the diluent is 1:0.05 to 1:10, preferably 1:0.1 to 1:8.5, further preferably 1:0.18 to 1:8.5, and more preferably 1:0.18125, 1:0.1875, 1:0.9875, 1:1, 1:1.1125, 1:1.125, 1:1.2375, 1:1.25, 1:1.3625, 1:1.375, 1:3.725, 1:3.75, 1:6.45, 1:6.95, 1:7.45, 1:7.95, 1:8.25, or 1:8.45;
in some embodiments, the weight ratio of the active ingredient to the disintegrant is 1:0.01 to 1:3, preferably 1:0.03 to 1:1.5, further preferably 1:0.03 to 1:0.6, and more preferably 1:0.0375, 1:0.0625, 1:0.075, 1:0.125, 1:0.15, 1:0.25, or 1:0.5;
in some embodiments, the weight ratio of the active ingredient to the lubricant is 1:0.001 to 1:2, preferably 1:0.006 to 1:0.1, and more preferably 1:0.00625, 1:0.0125, 1:0.025, 1:0.05, or 1:0.1. The present disclosure provides a pharmaceutical composition, comprising an active ingredient and lactose, microcrystalline cellulose, low-substituted hydroxypropyl cellulose and magnesium stearate.

The present disclosure provides a pharmaceutical composition, comprising an active ingredient and silica, lactose, microcrystalline cellulose, low-substituted hydroxypropyl cellulose and magnesium stearate.

The present disclosure provides a pharmaceutical composition, comprising an active ingredient and silica, talc, lactose, microcrystalline cellulose, low-substituted hydroxypropyl cellulose and magnesium stearate.

The present disclosure provides a pharmaceutical composition, comprising an active ingredient and microcrystalline cellulose, mannitol, crospovidone and magnesium stearate.

The present disclosure provides a pharmaceutical composition, comprising an active ingredient and silica, magnesium silicate, microcrystalline cellulose, mannitol, crospovidone and magnesium stearate.

The present disclosure provides a pharmaceutical composition, comprising an active ingredient and pregelatinized starch, lactose, sodium carboxymethyl starch and talc.

The present disclosure provides a pharmaceutical composition, comprising an active ingredient and silica, pregelatinized starch, lactose, sodium carboxymethyl starch and talc.

The present disclosure provides a pharmaceutical composition, comprising an active ingredient and microcrystalline cellulose, lactose, croscarmellose sodium and magnesium stearate.

The present disclosure provides a pharmaceutical composition, comprising an active ingredient and silica, microcrystalline cellulose, lactose, croscarmellose sodium and magnesium stearate.

The present disclosure provides a pharmaceutical composition, comprising an active ingredient and lactose, microcrystalline cellulose, croscarmellose sodium and magnesium stearate.

The present disclosure provides a pharmaceutical composition, comprising an active ingredient and lactose, croscarmellose sodium and magnesium stearate.

The present disclosure provides a pharmaceutical composition, comprising an active ingredient and microcrystalline cellulose, croscarmellose sodium and magnesium stearate.

The present disclosure provides a pharmaceutical composition, comprising an active ingredient and mannitol, low-substituted carboxymethyl cellulose sodium and talc.

The present disclosure provides a pharmaceutical composition, comprising an active ingredient and microcrystalline cellulose, low-substituted carboxymethyl cellulose sodium and talc.

The present disclosure provides a pharmaceutical composition, comprising an active ingredient and mannitol, microcrystalline cellulose, low-substituted carboxymethyl cellulose sodium and talc.

The present disclosure provides a pharmaceutical composition, comprising an active ingredient and pregelatinized starch, sodium carboxymethyl starch and magnesium stearate.

The present disclosure provides a pharmaceutical composition, comprising an active ingredient and dextrin, sodium carboxymethyl starch and magnesium stearate.

The present disclosure provides a pharmaceutical composition, comprising an active ingredient and pregelatinized starch, dextrin, sodium carboxymethyl starch and magnesium stearate.

The present disclosure provides a pharmaceutical composition, comprising an active ingredient and calcium phosphate, crospovidone and talc.

The present disclosure provides a pharmaceutical composition, comprising an active ingredient and sucrose, crospovidone and talc.

The present disclosure provides a pharmaceutical composition, comprising an active ingredient and calcium phosphate, sucrose, crospovidone and talc. The present disclosure provides a pharmaceutical composition, comprising 10 to 80 w/w %, 10 to 60 w/w %, 10 to 45 w/w %, 10 to 40 w/w %, 5 to 35 w/w %, 5 to 20 w/w %, 10%, 20%, 40%, or 80% of an active ingredient.

The present disclosure provides a pharmaceutical composition, comprising 10 to 85 w/w %, 10 to 80 w/w %, 10 to 70 w/w %, 10 to 60 w/w %, 10 to 50 w/w %, 10 to 40 w/w %, 10 to 30 w/w %, 14.5%, 15%, 39.5%, 40%, 44.5%, 45%, 49.5%, 50%, 54.5%, 55%, 64.5%, 69.5%, 74.5%, 75%, 79.5%, 80%, 82.5%, or 84.5% of a diluent.

The present disclosure provides a pharmaceutical composition, comprising 1 to 8 w/w %, 1 to 5 w/w %, 1 to 3 w/w %, 3 to 5 w/w %, 5 to 8 w/w %, 3 w/w %, 5 w/w %, or 8 w/w % of a disintegrant.

The present disclosure provides a pharmaceutical composition, comprising 0.1 to 2 w/w %, 0.1 to 1.5 w/w %, 0.1 to 1 w/w %, 0.1 to 0.5 w/w %, 0.5-2 w/w %, 0.5 to 1 w/w %, 0.5 w/w %, 1 w/w %, or 2 w/w % of a lubricant.

The present disclosure provides a pharmaceutical composition, comprising 2 to 35 w/w %, 2 to 30 w/w %, 2 to 25 w/w %, 2 to 20 w/w %, 2 to 15 w/w %, 2 to 10 w/w %, 2 to 5 w/w %, 5 to 35 w/w %, 5 to 30 w/w %, 5 to 25 w/w %, 5 to 20 w/w %, 5 to 15 w/w %, 5 to 10 w/w %, 2 w/w %, 5 w/w %, 10 w/w %, 15 w/w %, or 20 w/w % of an infiltrating agent.

The pharmaceutical composition according to any one aspect of the present disclosure comprises the active ingredients and the non-active ingredients of any one of the preceding embodiments, including:
an active ingredient;
a diluent, wherein the diluent is selected from one or more of pregelatinized starch, lactose, microcrystalline cellulose, mannitol, dextrin, calcium phosphate or sucrose;
a disintegrant, wherein the disintegrant is selected from one or more of croscarmellose sodium, sodium carboxymethyl starch, crospovidone or low-substituted hydroxypropyl cellulose;
a lubricant, wherein the lubricant is selected from one or more of magnesium stearate, talc or colloidal silica;
optionally, the pharmaceutical composition further comprises an infiltrating agent, wherein the infiltrating agent is selected from one or more of silica, talc, and magnesium silicate.

The present disclosure provides a pharmaceutical composition, comprising 5 to 35 w/w % of an active ingredient, 60 to 85 w/w % of a diluent, 1 to 8 w/w % of a disintegrant, and 0.1 to 2 w/w % of a lubricant.

The present disclosure provides a pharmaceutical composition, comprising 5 to 20 w/w % of an active ingredient, 70 to 85 w/w % of a diluent, 1 to 8 w/w % of a disintegrant, and 0.1 to 2 w/w % of a lubricant.

The present disclosure provides a pharmaceutical composition, comprising 10 to 80 w/w % of an active ingredient, 2 to 35 w/w % of an infiltrating agent, 15 to 85 w/w % of a diluent, 1 to 10 w/w % of a disintegrant, and 0.1 to 5 w/w % of a lubricant.

The present disclosure provides a pharmaceutical composition, comprising 10 to 60 w/w % of an active ingredient, 2 to 35 w/w % of an infiltrating agent, 25 to 80 w/w % of a diluent, 1 to 10 w/w % of a disintegrant, and 0.1 to 5 w/w % of a lubricant.

The present disclosure provides a pharmaceutical composition, comprising 10 to 45 w/w % of an active ingredient, 2 to 25 w/w % of an infiltrating agent, 30 to 70 w/w % of a diluent, 1 to 10 w/w % of a disintegrant, and 0.1 to 5 w/w % of a lubricant.

The present disclosure provides a pharmaceutical composition, comprising 10 to 40 w/w % of an active ingredient, 5 to 20 w/w % of an infiltrating agent, 35 to 60 w/w % of a diluent, 1 to 10 w/w % of a disintegrant, and 0.1 to 5 w/w % of a lubricant. The present disclosure provides a pharmaceutical composition, comprising 10 w/w % of an active ingredient, 24.5 w/w % of lactose, 60 w/w % of microcrystalline cellulose, 5 w/w % of low-substituted hydroxypropyl cellulose, and 0.5 w/w % of magnesium stearate.

The present disclosure provides a pharmaceutical composition, comprising 10 w/w % of an active ingredient, 2 w/w % of silica, 24.5 w/w % of lactose, 58 w/w % of microcrystalline cellulose, 5 w/w % of low-substituted hydroxypropyl cellulose, and 0.5 w/w % of magnesium stearate.

The present disclosure provides a pharmaceutical composition, comprising 10 w/w % of an active ingredient, 5 w/w % of silica, 24.5 w/w % of lactose, 55 w/w % of microcrystalline cellulose, 5 w/w % of low-substituted hydroxypropyl cellulose, and 0.5 w/w % of magnesium stearate.

The present disclosure provides a pharmaceutical composition, comprising 10 w/w % of an active ingredient, 10 w/w % of silica, 24.5 w/w % of lactose, 50 w/w % of microcrystalline cellulose, 5 w/w % of low-substituted hydroxypropyl cellulose, and 0.5 w/w % of magnesium stearate.

The present disclosure provides a pharmaceutical composition, comprising 10 w/w % of an active ingredient, 10 w/w % of silica, 5 w/w % of talc, 24.5 w/w % of lactose, 45 w/w % of microcrystalline cellulose, 5 w/w % of low-substituted hydroxypropyl cellulose, and 0.5 w/w % of magnesium stearate.

The present disclosure provides a pharmaceutical composition, comprising 10 w/w % of an active ingredient, 10 w/w % of silica, 10 w/w % of talc, 24.5 w/w % of lactose, 40 w/w % of microcrystalline cellulose, 5 w/w % of low-substituted hydroxypropyl cellulose, and 0.5 w/w % of magnesium stearate.

The present disclosure provides a pharmaceutical composition, comprising 20 w/w % of an active ingredient, 29.5 w/w % of mannitol, 45 w/w % of microcrystalline cellulose, 5 w/w % of crospovidone, and 0.5 w/w % of magnesium stearate.

The present disclosure provides a pharmaceutical composition, comprising 40 w/w % of an active ingredient, 19.5 w/w % of mannitol, 35 w/w % of microcrystalline cellulose, 5 w/w % of crospovidone, and 0.5 w/w % of magnesium stearate.

The present disclosure provides a pharmaceutical composition, comprising 80 w/w % of an active ingredient, 4.5 w/w % of mannitol, 10 w/w % of microcrystalline cellulose, 5 w/w % of crospovidone, and 0.5 w/w % of magnesium stearate.

The present disclosure provides a pharmaceutical composition, comprising 40 w/w % of an active ingredient, 3 w/w % of silica, 2 w/w % of magnesium silicate, 19.5 w/w % of mannitol, 30 w/w % of microcrystalline cellulose, 5 w/w % of crospovidone, and 0.5 w/w % of magnesium stearate.

The present disclosure provides a pharmaceutical composition, comprising 40 w/w % of an active ingredient, 6 w/w % of silica, 4 w/w % of magnesium silicate, 19.5 w/w % of mannitol, 25 w/w % of microcrystalline cellulose, 5 w/w % of crospovidone, and 0.5 w/w % of magnesium stearate.

The present disclosure provides a pharmaceutical composition, comprising 40 w/w % of an active ingredient, 10 w/w % of silica, 5 w/w % of magnesium silicate, 19.5 w/w % of mannitol, 20 w/w % of microcrystalline cellulose, 5 w/w % of crospovidone, and 0.5 w/w % of magnesium stearate.

The present disclosure provides a pharmaceutical composition, comprising 20 w/w % of an active ingredient, 40 w/w % of pregelatinized starch, 35 w/w % of lactose, 3 w/w % of sodium carboxymethyl starch, and 2 w/w % of talc.

The present disclosure provides a pharmaceutical composition, comprising 40 w/w % of an active ingredient, 30 w/w % of pregelatinized starch, 25 w/w % of lactose, 3 w/w % of sodium carboxymethyl starch, and 2 w/w % of talc.

The present disclosure provides a pharmaceutical composition, comprising 80 w/w % of an active ingredient, 10 w/w % of pregelatinized starch, 5 w/w % of lactose, 3 w/w % of sodium carboxymethyl starch, and 2 w/w % of talc.

The present disclosure provides a pharmaceutical composition, comprising 40 w/w % of an active ingredient, 5 w/w % of silica, 30 w/w % of pregelatinized starch, 20 w/w % of lactose, 3 w/w % of sodium carboxymethyl starch, and 2 w/w % of talc.

The present disclosure provides a pharmaceutical composition, comprising 40 w/w % of an active ingredient, 10 w/w % of silica, 25 w/w % of pregelatinized starch, 20 w/w % of lactose, 3 w/w % of sodium carboxymethyl starch, and 2 w/w % of talc.

The present disclosure provides a pharmaceutical composition, comprising 40 w/w % of an active ingredient, 15 w/w % of silica, 20 w/w % of pregelatinized starch, 20 w/w % of lactose, 3 w/w % of sodium carboxymethyl starch, and 2 w/w % of talc.

The present disclosure provides a pharmaceutical composition, comprising 20 w/w % of an active ingredient, 45 w/w % of microcrystalline cellulose, 29.5 w/w % of lactose, 5 w/w % of croscarmellose sodium, and 0.5 w/w % of magnesium stearate.

The present disclosure provides a pharmaceutical composition, comprising 40 w/w % of an active ingredient, 35 w/w % of microcrystalline cellulose, 19.5 w/w % of lactose, 5 w/w % of croscarmellose sodium, and 0.5 w/w % of magnesium stearate.

The present disclosure provides a pharmaceutical composition, comprising 80 w/w % of an active ingredient, 10 w/w % of microcrystalline cellulose, 4.5 w/w % of lactose, 5 w/w % of croscarmellose sodium, and 0.5 w/w % of magnesium stearate.

The present disclosure provides a pharmaceutical composition, comprising 40 w/w % of an active ingredient, 5 w/w % of silica, 30 w/w % of microcrystalline cellulose, 19.5 w/w % of lactose, 5 w/w % of croscarmellose sodium, and 0.5 w/w % of magnesium stearate.

The present disclosure provides a pharmaceutical composition, comprising 40 w/w % of an active ingredient, 10 w/w % of silica, 25 w/w % of microcrystalline cellulose, 19.5 w/w % of lactose, 5 w/w % of croscarmellose sodium, and 0.5 w/w % of magnesium stearate.

The present disclosure provides a pharmaceutical composition, comprising 40 w/w % of an active ingredient, 15 w/w % of silica, 20 w/w % of microcrystalline cellulose, 19.5 w/w % of lactose, 5 w/w % of croscarmellose sodium, and 0.5 w/w % of magnesium stearate.

The present disclosure provides a pharmaceutical composition, comprising 10 w/w % of an active ingredient, 22 w/w % of lactose, 62 w/w % of microcrystalline cellulose, 5 w/w % of croscarmellose sodium, and 1 w/w % of magnesium stearate.

The present disclosure provides a pharmaceutical composition, comprising 25 w/w % of an active ingredient, 30 w/w % of mannitol, 40 w/w % of microcrystalline cellulose, 3 w/w % of low-substituted carboxymethyl cellulose sodium, and 2 w/w % of talc.

The present disclosure provides a pharmaceutical composition, comprising 25 w/w % of an active ingredient, 37.5 w/w % of pregelatinized starch, 35 w/w % of dextrin, 1.5 w/w % of sodium carboxymethyl starch, and 1 w/w % of magnesium stearate.

The present disclosure provides a pharmaceutical composition, comprising 33 w/w % of an active ingredient, 27 w/w % of calcium phosphate, 38.33 w/w % of sucrose, 1 w/w % of crospovidone, and 0.67 w/w % of talc. In any one of the above-mentioned pharmaceutical compositions, the active ingredient is selected from a compound of formula (I), or a stereoisomer or a pharmaceutically acceptable salt thereof.

In one aspect, the present disclosure provides a pharmaceutical formulation, comprising any one of the above-mentioned pharmaceutical compositions;
in some embodiments, the amount of the active ingredient in a unit pharmaceutical formulation is from 1 mg to 100 mg; in some embodiments, the amount of the active ingredient in a unit pharmaceutical formulation is 5 mg, 10 mg, 20 mg, 25 mg, 30 mg, 40 mg, 50 mg, 60 mg, 70 mg, 80 mg, 90 mg, or 100 mg.

In some embodiments, the formulation form of the pharmaceutical formulation is selected from a tablet, a granule, a capsule, a dry suspension, an oral solution, a soft capsule, and an emulsion; in some embodiments, the formulation form of the pharmaceutical formulation is selected from a tablet, a granule, a capsule, and a soft capsule.

In one aspect, the present disclosure provides the use of any one of the above-mentioned pharmaceutical compositions or any one of the above-mentioned pharmaceutical formulations in the preparation of a medicament for treating an AAK1-induced related disease by inhibition or degradation of AAK1, wherein the disease is pain, preferably inflammatory pain, postoperative pain, trigeminal neuralgia, acute postherpetic neuralgia and postherpetic neuralgia, diabetic peripheral neuralgia, causalgia, occipital neuralgia, fibromyalgia, phantom limb pain, burn pain and other forms of neuralgia, neuropathy and spontaneous pain syndrome.

The preparation process of the pharmaceutical composition or the pharmaceutical formulation of the present disclosure is one or more of direct mixing, wet granulation, dry granulation, fluidized bed granulation, spray drying, freeze drying, hot melt extrusion, and pellet coating, preferably direct mixing, wet granulation, fluidized bed granulation, dry granulation and spray drying, and more preferably direct mixing and dry granulation.

In some embodiments, the preparation process is one or more of direct mixing, wet granulation, dry granulation, fluidized bed granulation, spray drying, freeze drying, hot melt extrusion, pellet coating, and extrusion spheronization, preferably spray drying and hot melt extrusion;
in some embodiments, the preparation process includes dry granulation for tableting, direct powder compression for tableting, and direct powder filling into capsules.

In some embodiments, the preparation process of the pharmaceutical composition further involves pretreatment of the active ingredient, wherein the pretreatment is selected from one or more of pulverization, solid dispersion, and nano-grinding.

Unless stated to the contrary, the terms used in both the description and the claims have the following meanings:
The “pharmaceutically acceptable salt” refers to a salt that is safe, non-toxic, and neither biologically nor otherwise undesirable, is pharmaceutically acceptable for veterinary use and human pharmaceutical use, and has the desired pharmacological activity.

The term “stereoisomer” refers to an isomer produced as a result of different spatial arrangement of atoms in molecules, including cis-trans isomers, enantiomers and conformational isomers.

“Optional” or “optionally” or “alternative” or “alternatively” means that the events or conditions subsequently described may but not necessarily occur, and the description includes the case where the events or conditions occur and do not occur. For example, “heterocyclyl alternatively substituted with alkyl” means that the alkyl may but not necessarily exist, and the description includes the case where the heterocyclyl is substituted with alkyl and the case where the heterocyclyl is not substituted with alkyl.

### BRIEF DESCRIPTION OF THE DRAWINGS

FIG. 1 is a time-mechanical pain threshold (MPT) curve of the mice in Example 5.

FIG. 2 is a dissolution curve of the tablet in Example 7.

FIG. 3 is a dissolution curve of the capsule in Example 7.

### DETAILED DESCRIPTION

The technical problems to be solved, technical solutions and beneficial effects of the present disclosure are described below in conjunction with examples. It should be understood that the specific examples described herein are merely used to explain the present disclosure, but are not intended to limit the present disclosure.

A compound of general formula (I) is prepared according to the following synthesis scheme:

### Example 1: Preparation of Active Ingredients

### Intermediate 1

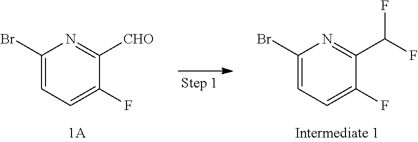 [SMILES: O=Cc1nc(Br)ccc1F ; Fc1ccc(Br)nc1C(F)F]

### Step 1:

Raw material 1A (10 g, 49 mmol) was dissolved in 200 mL of dichloromethane, and the mixture was cooled to −20° C. DAST (11.7 mL, 88 mmol) was added and the resulting mixture was slowly warmed to room temperature and reacted for 5 h. Upon complete depletion of raw materials monitored by TLC, the reaction was quenched with saturated aqueous sodium bicarbonate solution. The resulting reaction mixture was extracted with ethyl acetate and the organic phase was subjected to rotary evaporation. Then the residue was purified by silica gel column (petroleum ether:ethyl acetate=20:1) to obtain the target compound intermediate 1 (9.8 g, 89%).

1H NMR (400 MHz, CDCl3) δ7.65-7.58 (m, 1H), 7.46-7.40 (m, 1H), 6.85-6.56 (m, 1H).

### Intermediate 2

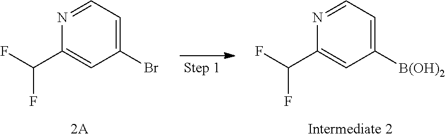 [SMILES: FC(F)c1cc(Br)ccn1 ; OB(O)c1ccnc(C(F)F)c1]

### Step 1:

2A (5 g, 24 mmol), Xphos PdG2 (189 mg, 0.24 mmol, CAS: 1310584-14-5), Xphos (229 mg, 0.48 mmol, CAS 564483-18-7), bis(pinacolato)diboron (9.14 g, 36 mmol) and KOAc (7.07 g, 72 mmol) were added to a flask. After nitrogen replacement, 200 mL of ethanol was added and the mixture was heated to 80° C. and reacted for 5 h. Upon complete depletion of raw materials monitored by TLC, water was added to quench the reaction. The system was subjected to rotary evaporation to remove ethanol and then extracted with ethyl acetate. The organic phase was subjected to rotary evaporation to obtain intermediate 2 (5.1 g).

LC-MS (ESI): m/z=174.1 [M+H]+.

### Intermediate 3

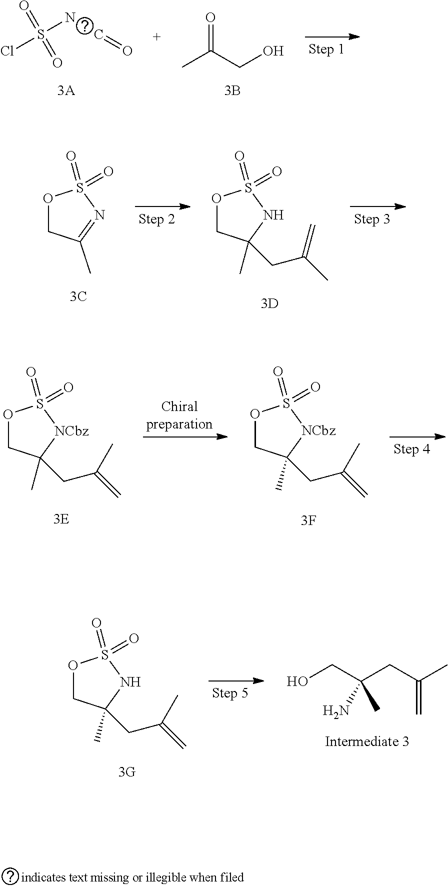

### Step 1:

Under nitrogen atmosphere, chlorosulfonyl isocyanate (62 mL) was added to a three-necked round bottom flask, then 200 mL of dichloromethane was added, and the system was cooled to 0° C. 27 mL of formic acid was dissolved in 50 mL of dichloromethane. The mixture was slowly added to the system while the temperature was controlled at 0° C. After 30 minutes, the mixture was warmed to room temperature and stirred overnight. Hydroxyacetone (36.3 mL) and pyridine (58 mL) were dissolved in 1000 mL of dichloromethane. At 0° C., the mixture was slowly added dropwise to the system. After the addition was completed, the system was warmed to room temperature and stirred overnight. The system was subjected to rotary evaporation to remove the organic solvent and the residue was purified by silica gel column (eluent: dichloromethane) to obtain the title compound 3C (36 g, 56%).

1H NMR (400 MHz, CDCl3) δ 5.06 (s, 2H), 2.42 (s, 3H).

### Step 2:

Under nitrogen atmosphere, 3C (36 g, 267 mmol) was dissolved in 800 mL of methyl tert-butyl ether. The system was cooled to 0° C. and then a solution of 2-methylallylmagnesium chloride in tetrahydrofuran (0.55 L, 0.5 M) was added dropwise. Upon complete depletion of raw materials monitored by TLC, the reaction was quenched by the addition of saturated aqueous ammonium chloride solution, and the resulting mixture was extracted with ethyl acetate and then subjected to rotary evaporation. The residue was purified by silica gel column to obtain the title compound 3D (43 g, 84%).

1H NMR (400 MHz, CDCl3) δ 5.06-5.01 (m, 1H), 4.85-4.83 (m, 1H), 4.59 (s, 1H), 4.38 (d, 1H), 4.27 (d, 1H), 2.57-2.50 (m, 1H), 2.42-2.29 (m, 1H), 1.84 (s, 3H), 1.46 (s, 3H).

### Step 3:

Under nitrogen, 3D (1.91 g, 10 mmol) was dissolved in 50 mL of tetrahydrofuran. A solution of 1 M potassium tert-butoxide in tetrahydrofuran (15 mL) was added, followed by CbzCl (2.1 mL, 15 mmol). Upon complete depletion of raw materials monitored by TLC, the reaction was quenched by the addition of saturated aqueous ammonium chloride solution. The system was subjected to rotary evaporation to remove tetrahydrofuran, extracted with ethyl acetate, and then subjected to rotary evaporation. The residue was then purified by silica gel column (petroleum ether:ethyl acetate=10:1) to obtain the title compound 3E (2.6 g, 80%).

LC-MS (ESI): m/z=343.0 [M+NH4]+.

120 g of 3E was subjected to chiral preparation to obtain the target compound 3F (55 g).

Preparation method: instrument: Waters SFC 150 Mgm, column: DAICEL CHIRALPAK OJ (250 mm×50 mm, 10 μm); mobile phase: A for CO2 and B for MeOH (BASE); gradient: 10% B; flow rate: 130 mL/min, back pressure: 100 bar; column temperature: 35° C.; wavelength: 220 nm; cycle time: 4.5 min; sample preparation: sample concentration: 157.5 mg/ml, ethanol solution; sample injection: 0.8 ml/injection. After separation, the fractions were dried on a rotary evaporator at the bath temperature of 40° C. to obtain compound 3F (retention time: 0.680 minutes).

### Step 4:

Compound 3F (5 g, 15.4 mmol) was dissolved in 500 mL of methanol and 50 mg of 10% palladium on carbon catalyst was added. The mixture was subjected to hydrogen replacement. Upon the disappearing of fluorescence monitored by TLC, the system was filtered by suction to remove palladium on carbon from the system. The resulting filtrate was subjected to rotary evaporation to obtain the title compound 3G (crude), which was directly used in the next step.

### Step 5:

Compound 3G was dissolved in 150 mL of tetrahydrofuran. At 0° C., lithium aluminum hydride (1.8 g, 47.4 mmol) was added in portions and the mixture was warmed to room temperature and stirred overnight. Water (1.8 mL), 10% aqueous sodium hydroxide solution (3.6 mL) and water (5.4 mL) were added and the mixture was stirred for 1 h. The resulting reaction mixture was filtered by suction to remove the solid. The resulting filtrate was subjected to rotary evaporation to obtain intermediate 3 (crude product), which was directly used in the next reaction.

LC-MS (ESI): m/z=130.1 [M+H]+.

### Intermediate 4

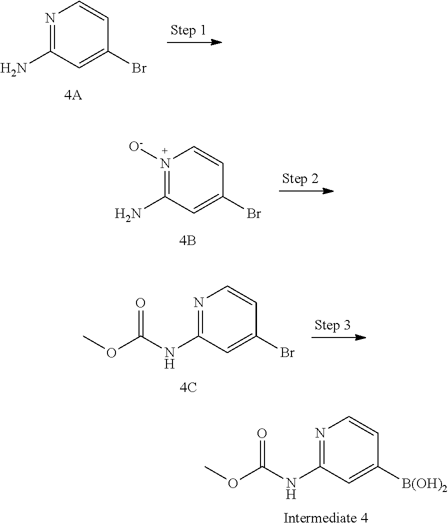

### Step 1:

Compound 4A (10 g, 57.8 mmol) was dissolved in 200 mL of acetone. At room temperature, m-chloroperoxybenzoic acid (11 g, 63.6 mmol) was dissolved in 200 mL of acetone and then the resulting mixture was added. The reaction mixture was stirred for 5 min to produce a large quantity of a solid. The solid was collected by suction filtration, washed with acetone, and dried to obtain compound 4B (crude product, 10.7 g, 98%).

LC-MS (ESI): m/z=189.0 and 191.0 [M+H]+.

### Step 2:

Compound 4B (crude, 10.7 g) was dissolved in trimethyl orthoformate (200 mL) and 1.25 mL of boron trifluoride diethyl etherate was added. The system was heated to 105° C. and reacted overnight. The organic phase in the system was subjected to rotary evaporation, and then the residue was separated by column chromatography to obtain compound 4C (9.1 g, 69%).

LC-MS (ESI): m/z=231.0 and 233.0 [M+H]+.

### Step 3:

Compound 4C (3.5 g, 15.1 mmol), Xphos PdG2 (600 mg, 0.76 mmol, CAS: 1310584-14-5), Xphos (700 mg, 1.47 mmol, CAS 564483-18-7), potassium acetate (4.5 g, 45.8 mmol) and bis(pinacolato)diboron (6 g, 23.6 mmol) were dissolved in 250 mL of ethanol in a round bottom flask. The system was subjected to nitrogen replacement, warmed to 80° C. and reacted overnight. The system was subjected to rotary evaporation to remove ethanol and then extracted with ethyl acetate to obtain the title compound intermediate 4 (4 g).

LC-MS (ESI): m/z=197.1 [M+H]+.

### Intermediate 5

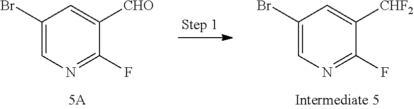 [SMILES: O=Cc1cc(Br)cnc1F ; Fc1ncc(Br)cc1C(F)F]

### Step 1:

Raw material 5A (5.00 g, 24.51 mmol) was dissolved in 100 mL of dichloromethane, and the mixture was cooled to −20° C. DAST (6.5 mL, 49.02 mmol) was added, and the resulting mixture was slowly warmed to room temperature and reacted for 2 h. Upon complete depletion of raw materials monitored by TLC, the reaction was quenched with saturated aqueous sodium bicarbonate solution and extracted with dichloromethane. The organic phase was subjected to rotary evaporation and the residue was purified by silica gel column (petroleum ether:ethyl acetate=20:1) to obtain intermediate 5 (5.00 g, 90.27%).

1H NMR (400 MHz, CDCl3) δ 8.40 (dd, 1H), 8.15 (dt, 1H), 6.95-6.67 (m, 1H).

### 1.1 Preparation of Compound 1

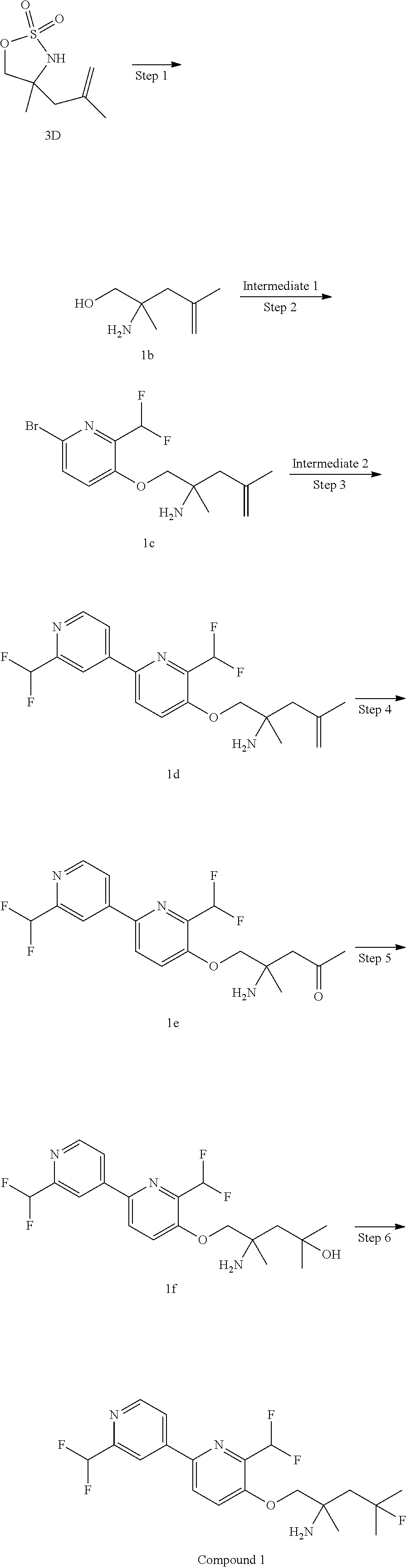

### Step 1:

3D (8 g, 42 mmol) was dissolved in 500 mL of tetrahydrofuran. The system was cooled to 0° C. and lithium aluminum hydride (3.99 g, 105 mmol) was slowly added. Then the mixture was warmed to room temperature and reacted for 6 h. Water (4 mL), 8 M aqueous NaOH solution, and water (12 mL) were sequentially added and the mixture was stirred for 1 h. The resulting mixture was filtered by suction to remove the solid and the resulting filtrate was subjected to rotary evaporation to obtain the target compound 1b (crude product, 9 g), which was directly used in the next step without purification.

LC-MS (ESI): m/z=130.2 [M+H]+.

### Step 2:

Crude product 1b (2 g) was added to a solution of potassium tert-butoxide in tetrahydrofuran (27 mL). At room temperature, the mixture was stirred for 5 min and then intermediate 1 (4 g, 18 mmol) was added. The resulting mixture was subjected to nitrogen replacement, then heated to 80° C. and reacted overnight. The organic phase in the system was subjected to rotary evaporation, and the residue was separated and purified by silica gel column chromatography (dichloromethane:methanol=10:1) to obtain the target compound 1c (1.1 g, 35%).

LC-MS (ESI): m/z=335.1 and 337.1 [M+H]+.

### Step 3:

Intermediate 2 (1.1 g, 3.3 mmol), 1c (880 mg, 5 mmol), potassium phosphate (9.2 g, 43 mmol), Xphos PdG2 (500 mg, 0.63 mmol, CAS: 1310584-14-5), and Xphos (650 mg, 1.36 mmol, CAS 564483-18-7) were added to a sealed tube, and 30 mL of tetrahydrofuran was added. After nitrogen replacement, the mixture was warmed to 80° C. and reacted for 5 h. Upon complete depletion of raw materials monitored by TLC, the system was filtered by suction to remove the solid, which was then washed with methanol. The filtrate was collected and subjected to rotary evaporation, and the residue was separated and purified by silica gel column chromatography (dichloromethane:methanol=10:1) to obtain the title compound 1d (360 mg, 29%).

LC-MS (ESI): m/z=384.2 [M+H]+.

¹H NMR (400 MHz, DMSO-d₆) δ 8.80-8.76 (m, 1H), 8.42-8.36 (m, 1H), 8.32 (s, 1H), 8.24-8.18 (m, 1H), 7.84-7.79 (m, 1H), 7.42-6.88 (m, 2H), 4.87 (s, 1H), 4.72 (s, 1H), 3.88 (s, 2H), 2.22 (s, 2H), 1.78 (s, 3H), 1.15 (s, 3H).

### Step 4:

1d (360 mg, 0.94 mmol) was dissolved in 20 mL of dichloromethane. The mixture was cooled to −60° C. and ozone was introduced. Upon complete depletion of raw materials monitored by TLC, 1 g of triphenylphosphine was added and the system was warmed to room temperature and stirred for 15 min. The organic phase was subjected to rotary evaporation and the residue was separated and purified by silica gel column chromatography (dichloromethane:methanol=10:1) to obtain the title compound 1e (300 mg, 83%).

LC-MS (ESI): m/z=386.2 [M+H]+.

### Step 5:

Under nitrogen atmosphere, 1e (300 mg, 0.78 mmol) was dissolved in 20 mL of tetrahydrofuran and the system was cooled to 0° C. A solution of methylmagnesium bromide in THF (1 mL, 3 M) was added and the mixture was slowly warmed to room temperature. Upon complete depletion of raw materials monitored by TLC, the reaction was quenched by the addition of saturated aqueous ammonium chloride solution, and extracted with dichloromethane. The organic phase was subjected to rotary evaporation to obtain the title compound if (240 mg, 0.6 mmol), which was directly used in the next step.

LC-MS (ESI): m/z=402.2 [M+H]+.

### Step 6:

Under nitrogen atmosphere, if (240 mg, 0.6 mmol) was dissolved in 15 mL of dichloromethane and the mixture was cooled to −78° C. DAST (0.4 mL, 2.8 mmol) was added and the system was slowly warmed to room temperature. Upon complete depletion of raw materials monitored by TLC, the reaction was quenched by the addition of saturated aqueous sodium bicarbonate solution and extracted with dichloromethane. The organic phase was subjected to rotary evaporation and then the resulting product was separated by HPLC and lyophilized to obtain the title compound 1 (110 mg, 42%).

LC-MS (ESI): m/z=404.2 [M+H]+.

¹H NMR (400 MHz, DMSO-d₆) δ 8.82-8.76 (m, 1H), 8.42-8.36 (m, 1H), 8.32 (s, 1H), 8.23-8.18 (m, 1H), 7.81-7.75 (m, 1H), 7.41-6.89 (m, 2H), 3.95 (s, 2H), 1.94-1.86 (m, 2H), 1.49-1.36 (m, 6H), 1.23 (s, 3H).

### 1.2 Preparation of Compounds 2 and 3

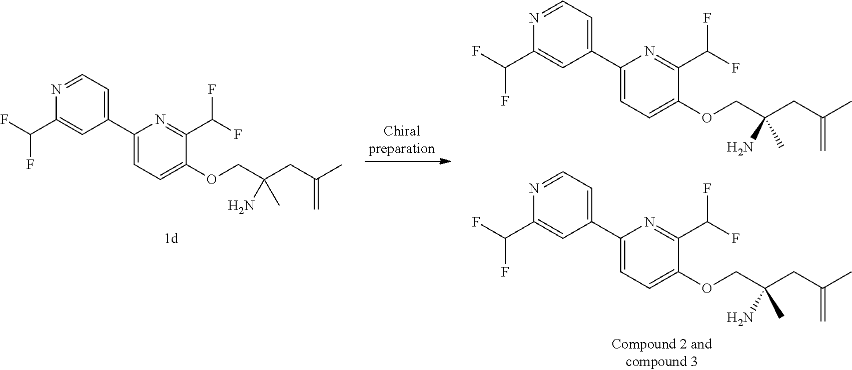 [SMILES: C=C(C)CC(C)(N)COc1ccc(-c2ccnc(C(F)F)c2)nc1C(F)F ; C=C(C)C[C@@](C)(N)COc1ccc(-c2ccnc(C(F)F)c2)nc1C(F)F ; C=C(C)C[C@](C)(N)COc1ccc(-c2ccnc(C(F)F)c2)nc1C(F)F]

1d (80 mg) was subjected to chiral resolution to obtain compound 2 (33.7 mg) and compound 3 (25.3 mg).

### Preparation Method:

instrument: SHIMADZU LC-20AP, column: DAICEL CHIRALPAK IG (250 mm×30 mm, 10 μm); mobile phase: A: n-hexane, B: ethanol (0.1% NH₃·H₂O); gradient: 8% B gradient elution; flow rate: 120 mL/min, column temperature: 25° C., wavelength: 254 nm, cycle time: 16 min; sample preparation: sample concentration: 1.5 mg/ml, ethanol solution; sample injection: 2 ml/injection. After separation, the fractions were dried on a rotary evaporator at the bath temperature of 40° C. to obtain P1 (retention time: 2.658 minutes, set to be compound 2) and P2 (retention time: 4.205 minutes, set to be compound 3).

### 1.3 Preparation of Compound 4

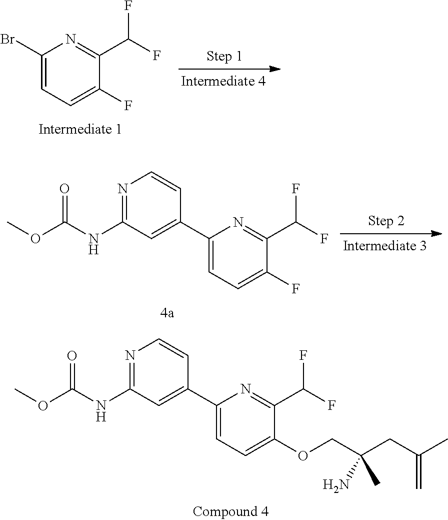 [SMILES: Fc1ccc(Br)nc1C(F)F ; COC(=O)Nc1cc(-c2ccc(F)c(C(F)F)n2)ccn1 ; C=C(C)C[C@](C)(N)COc1ccc(-c2ccnc(NC(=O)OC)c2)nc1C(F)F]

### Step 1:

Intermediate 4 (500 mg, 1.8 mmol), intermediate 1 (500 mg, 2.2 mmol), Xphos PdG2 (200 mg, 0.25 mmol, CAS: 1310584-14-5), Xphos (250 mg, 0.52 mmol, CAS 564483-18-7), and potassium phosphate (4.5 g, 21.2 mmol) were added to a sealed tube, and 20 mL of tetrahydrofuran was added. After nitrogen replacement, the system was warmed to 80° C. and reacted for 3 h. The resulting reaction mixture was mixed with silica gel and separated by column chromatography to obtain compound 4a (197 mg, 37%).

LC-MS (ESI): m/z=298.1 [M+H]+.

### Step 2:

Compound 4a (197 mg, 0.66 mmol), intermediate 3 (90 mg, 0.7 mmol) and 1 mL of potassium tert-butoxide (1 M in THF) were added to a sealed tube. After nitrogen replacement, the system was warmed to 80° C. and reacted for 3 h. The reaction solution was concentrated to dryness, and the residue was separated and purified by preparative chromatography to obtain the target compound 4 (30 mg, 11%).

LC-MS (ESI): m/z=407.1 [M+H]+.

¹H NMR (400 MHz, DMSO-d₆) δ 10.23 (s, 1H), 8.49 (s, 1H), 8.38-8.33 (m, 1H), 8.18-8.13 (m, 1H), 7.79-7.75 (m, 1H), 7.69-7.63 (m, 1H), 7.40-7.08 (m, 1H), 4.86 (s, 1H), 4.71 (s, 1H), 3.88 (s, 2H), 3.71 (s, 3H), 2.23 (s, 2H), 1.78 (s, 3H), 1.14 (s, 3H).

### 1.4 Preparation of Compound 5

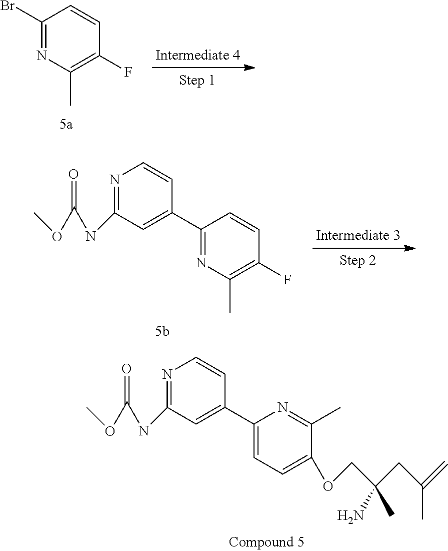

### Step 1:

5a (1.5 g, 7.89 mmol), intermediate 4 (2.3 g, 11.84 mmol), potassium phosphate (21.8 g, 102.57 mmol), Xphos PdG2 (1.24 g, 1.58 mmol, CAS: 1310584-14-5), and Xphos (1.5 g, 3.16 mmol, CAS 564483-18-7) were added to a sealed tube, and 60 mL of tetrahydrofuran was added. After nitrogen replacement, the mixture was warmed to 80° C. and reacted for 5 h. Upon complete depletion of raw materials monitored by TLC, the system was filtered by suction to remove the solid, which was then washed with methanol. The filtrate was collected and subjected to rotary evaporation, and the residue was purified by silica gel column (dichloromethane methanol=10:1) to obtain the title compound 5b (1.4 g, 68%).

LC-MS (ESI): m/z=262.0 [M+H]+.

### Step 2:

Intermediate 3 (495 mg, 3.83 mmol) was added to 15 mL of DMF solution. Under an ice bath, NaH (275 mg, 11.49 mmol) was added and the mixture was stirred for 10 min. Then compound 5b (1 g, 3.83 mmol) was added. After nitrogen replacement, the mixture was reacted at 0° C. for 1 h. The reaction was quenched by the addition of water and the mixture was then extracted with ethyl acetate. The organic phase was subjected to rotary evaporation and the residue was purified by column chromatography (dichloromethane:methanol=10:1) to obtain compound 5 (110 mg).

LC-MS (ESI): m/z=371.2 [M+H]+.

¹H NMR (400 MHz, DMSO-d₆) δ 10.16 (s, 1H), 8.47 (s, 1H), 8.29 (d, 1H), 7.80 (d, 1H), 7.62 (dd, 1H), 7.40 (d, 1H), 4.86 (s, 1H), 4.70 (s, 1H), 3.75 (s, 2H), 3.70 (s, 3H), 2.50 (s, 3H), 2.23 (s, 2H), 1.78 (s, 3H), 1.58 (s, 2H), 1.14 (s, 3H).

### 1.5 Preparation of Compound 6

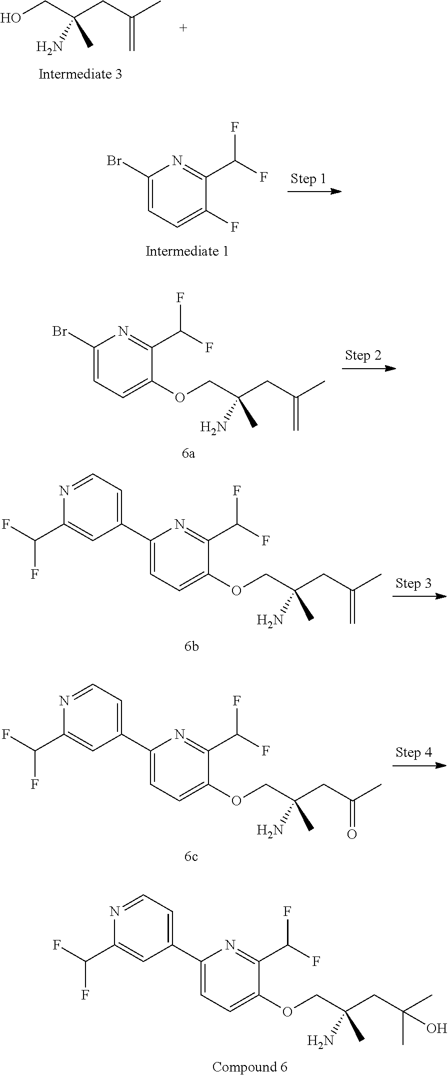

### Step 1:

Intermediate 3 (1 g 7.7 mmol) intermediate 1 (1.6 g 7.1 mmol) and 12 mL of potassium tert-butoxide (1 M in THF) were added to a sealed tube. After nitrogen replacement, the system was warmed to 80° C. and reacted for 3 h. The system was cooled to room temperature, mixed with silica gel, and separated by column chromatography (petroleum ether:ethyl acetate=1:1 to ethyl acetate) to obtain the target compound 6a (500 mg, 21%).

LC-MS (ESI): m/z=355.1 [M+H]+.

### Step 2:

Compound 6a (500 mg, 1.5 mmol), intermediate 2 (620 mg, 3.6 mmol), Xphos PdG2 (200 mg, 0.25 mmol, CAS: 1310584-14-5), Xphos (250 mg, 0.52 mmol, CAS 564483-18-7), and potassium phosphate (4.5 g, 21.2 mmol) were added to a sealed tube, and 20 mL of tetrahydrofuran was added. After nitrogen replacement, the system was warmed to 80° C. and reacted for 3 h. The resulting reaction mixture was mixed with silica gel and separated by column chromatography (petroleum ether:ethyl acetate 1:1 to ethyl acetate) to obtain compound 6b (350 mg, 61%).

LC-MS (ESI): m/z=384.2 [M+H]+.

### Step 3:

Compound 6b (350 mg, 0.91 mmol) was dissolved in 20 mL of dichloromethane. The system was cooled to −78° C. and ozone was introduced. Upon complete depletion of raw materials monitored by TLC, excess triphenylphosphine was added and the mixture was slowly warmed to room temperature, mixed with silica gel, and separated by column chromatography (petroleum ether:ethyl acetate=1:1 to ethyl acetate) to obtain compound 6c (310 mg, 89%).

LC-MS (ESI): m/z=386.1 [M+H]+.

### Step 4:

Compound 6c (160 mg, 0.42 mmol) was dissolved in 10 mL of tetrahydrofuran and the mixture was subjected to nitrogen replacement. At 0° C., 1.4 mL of methylmagnesium chloride (3 M, dissolved in THF) was added. The mixture was slowly warmed to room temperature, and the reaction was quenched by the addition of saturated aqueous ammonium chloride solution. The organic phase in the system was subjected to rotary evaporation, extracted with dichloromethane and then subjected to rotary evaporation. The residue was purified by preparative chromatography and then lyophilized to obtain compound 6 (30 mg, 18%).

LC-MS (ESI): m/z=402.2 [M+H]+.

¹H NMR (400 MHz, DMSO-d₆) δ 8.81-8.78 (m, 1H), 8.42-8.37 (m, 1H), 8.32 (s, 1H), 8.23-8.19 (m, 1H), 7.81-7.74 (m, 1H), 7.41-6.88 (m, 2H), 4.02-3.91 (m, 2H), 1.71 (d, 1H), 1.60 (d, 1H), 1.27 (s, 3H), 1.23 (s, 3H), 1.16 (s, 3H).

### 1.6 Preparation of Compound 7

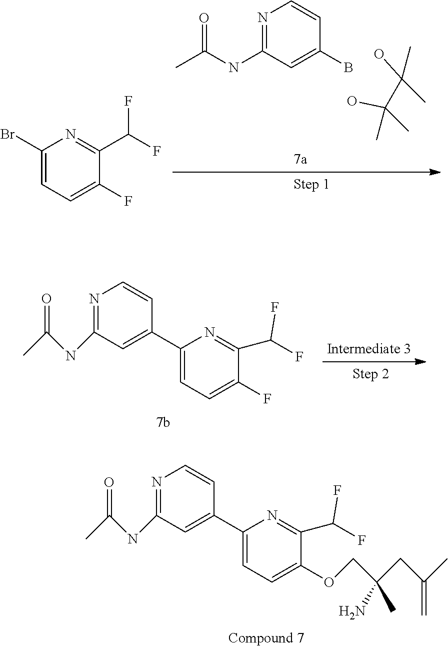

### Step 1:

7a (900 mg), intermediate 1 (633 mg, 2.8 mmol), Xphos PdG2 (250 mg, 0.32 mmol), Xphos (500 mg, 1.05 mmol), and potassium phosphate (6.0 g, 28.3 mmol) were added to a sealed tube, and 30 mL of tetrahydrofuran was added. After nitrogen replacement, the system was warmed to 80° C. and reacted for 3 h. The resulting reaction mixture was mixed with silica gel and separated by column chromatography to obtain compound 7b (428 mg, 54%).

LC-MS (ESI): m/z=282.2 [M+H]+.

### Step 2:

Compound 7b (200 mg, 0.71 mmol), intermediate 3 (100 mg, 0.77 mmol) and 2.5 mL of potassium tert-butoxide (1 M in THF) were added to a sealed tube. After nitrogen replacement, the system was warmed to 80° C. and reacted for 3 h. The reaction mixture was separated and purified by preparative chromatography to obtain compound 7 (89 mg, 32%).

LC-MS (ESI): m/z=391.1 [M+H]+.

¹H NMR (400 MHz, DMSO-d₆) δ 10.54 (s, 1H), 8.68 (s, 1H), 8.41-8.37 (m, 1H), 8.16-8.10 (m, 1H), 7.81-7.75 (m, 1H), 7.72-7.67 (m, 1H), 7.38-7.07 (m, 1H), 4.86 (s, 1H), 4.71 (s, 1H), 3.88 (s, 2H), 2.23 (s, 2H), 2.12 (s, 3H), 1.78 (s, 3H), 1.14 (s, 3H).

### 1.7 Preparation of Compound 8

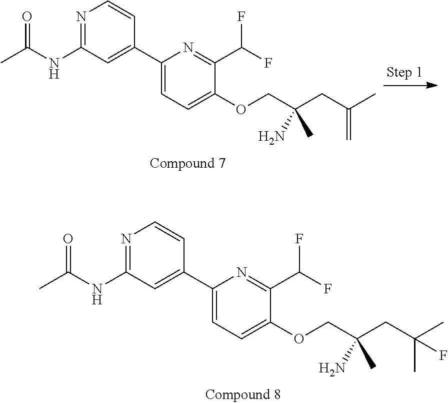

### Step 1:

Ferric nitrate nonahydrate (133 mg, 0.33 mmol) was dissolved in water (3 mL) and the mixture was subjected to nitrogen replacement and then cooled to 0° C. Selective fluorination reagent (117 mg, 0.33 mmol) and 3 mL of acetonitrile were added. Compound 7 (35 mg, 0.09 mmol) was dissolved in 3 mL of acetonitrile and the resulting mixture was added to the system. The mixture was stirred for 5 min and then sodium borohydride (40 mg, 1.05 mmol) was added in portions. The resulting mixture was reacted for 30 min while the temperature was maintained at 0° C. Ammonia water (1 mL) was added to quench the reaction, followed by extraction with the mixed solvents of dichloromethane and methanol (10:1). After rotary evaporation, the residue was purified by preparative HPLC to obtain compound 8 (10 mg, 28%).

LC-MS (ESI): m/z=411.3 [M+H]⁺.

¹H NMR (400 MHz, DMSO-d₆) δ 10.55 (s, 1H), 8.69 (s, 1H), 8.43-8.35 (m, 1H), 8.18-8.10 (m, 1H), 7.78-7.73 (m, 1H), 7.72-7.65 (m, 1H), 7.40-7.05 (m, 1H), 3.94 (s, 2H), 2.12 (s, 3H), 1.95-1.86 (m, 2H), 1.50-1.36 (m, 6H), 1.23 (s, 3H).

### 1.8 Preparation of Compound 9

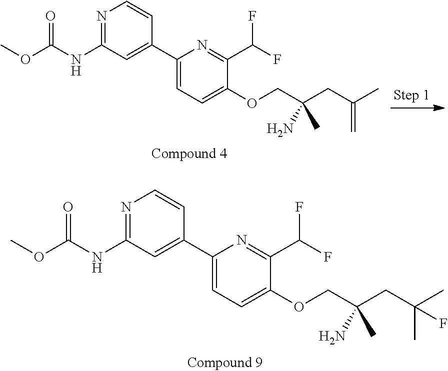

### Step 1:

Ferric nitrate nonahydrate (324 mg, 0.8 mmol) was dissolved in water (7 mL) and the mixture was subjected to nitrogen replacement and then cooled to 0° C. Selective fluorination reagent (284 mg, 0.8 mmol) and 7 mL of acetonitrile were added and compound 4 (81 mg, 0.2 mmol) was dissolved in 7 mL of acetonitrile. The resulting mixture was added to the system. The resulting mixture was stirred for 5 min and then sodium borohydride (100 mg, 2.6 mmol) was added in portions. The reaction mixture was reacted for 30 min while the temperature was maintained at 0° C. Ammonia water (2.5 mL) was added to quench the reaction, followed by extraction with the mixed solvents of dichloromethane:methanol (10:1). After rotary evaporation, the residue was purified by preparative HPLC to obtain compound 9 (9 mg, 11%).

LC-MS (ESI): m/z=427.2 [M+H]+.

¹H NMR (400 MHz, DMSO-d₆) δ 10.25 (s, 1H), 8.49 (s, 1H), 8.38-8.33 (m, 1H), 8.19-8.13 (m, 1H), 7.78-7.72 (m, 1H), 7.68-7.64 (m, 1H), 7.38-7.09 (m, 1H), 3.94 (s, 2H), 3.71 (s, 3H), 1.95-1.86 (m, 2H), 1.50-1.37 (m, 6H), 1.23 (s, 3H).

### 1.9 Preparation of Compound 10

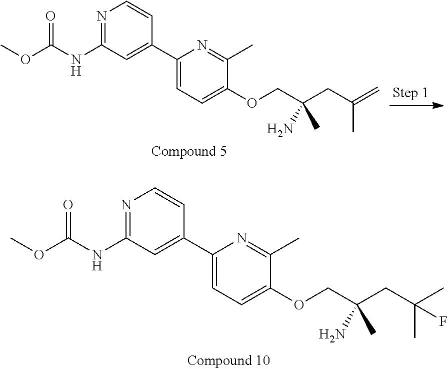

Ferric nitrate nonahydrate (88 mg, 0.22 mmol) was dissolved in water (2 mL). The mixture was ultra-sonicated for 5 min and cooled to 0° C. Then a solution of selective fluorination reagent (76 mg, 0.22 mmol) in 2 mL of acetonitrile was added, followed by a solution of compound 5 (20 mg, 0.05 mmol) in 2 mL of acetonitrile. Sodium borohydride (30 mg, 0.79 mmol) was then added in portions. The mixture was reacted for 1 h. When the reaction of the raw materials was completed as shown by LC-MS, the reaction mixture was diluted by the addition of water and then extracted with dichloromethane. The organic phases were combined, dried and concentrated to obtain a crude, which was separated and purified by preparative chromatography to obtain compound 10 (10 mg, 47%).

LC-MS (ESI): m/z=391.3[M+H]+.

¹H NMR (400 MHz, CD₃OD) δ 8.42 (s, 1H), 8.27 (d, 1H), 7.76 (d, 1H), 7.61 (d, 1H), 7.40 (d, 1H), 3.98 (s, 2H), 3.80 (s, 3H), 2.57 (s, 3H), 2.09 (s, 1H), 2.04 (s, 1H), 1.50 (d, 3H), 1.45 (d, 3H), 1.40 (s, 3H).

### Example 2: In Vitro AAK1 Enzyme Activity Assay

Compound stock solution (concentration: 10 mM, dissolved in DMSO) was diluted with DMSO to 0.2 mM and then diluted with DMSO in 5-fold gradient to obtain compound solutions with 10 concentrations. Subsequently, the compound solutions with different concentrations were diluted 50-fold in 1× kinase reaction buffer (containing 40 mM Tris, 20 mM MgCl₂, 0.1% BSA and 0.5 mM DTT) for later use. AAK1 (Signalchem, Cat #A01-11G-10) was diluted with 1× kinase reaction buffer to 2-fold the final concentration (final concentrations: 30 nM and 28 nM). AAK1 was added to a 384-well white plate at 2 μL/well, and the compounds were then added at 1 μL/well. The plate was sealed with a plate-sealing film, centrifuged at 1000 rpm for 30 seconds and then placed at room temperature for 10 minutes. A mixed solution of ATP (Promega, Cat #V914B) and substrate Micro2 (GenScript, Cat #PE0890) was formulated at 4-fold the final concentration (for AAK1, the corresponding final concentrations of ATP: 15 μM and 5 μM, and the corresponding final concentration of Micro2: 0.1 mg/mL). To the reaction plate was added the mixed solution of ATP and the substrate at 1 μL/well. The plate was sealed with a plate-sealing film and centrifuged at 1000 rpm for 30 seconds. The reaction was carried out at room temperature for 60 minutes (AAK1). ADP-Glo (Promega, Cat #V9102) was transferred to the 384-well plate at 4 μL/well and centrifugation was carried out at 1000 rpm for 1 minute. The mixture was incubated at 25° C. for 40 minutes. A detection solution was transferred to the 384-well plate at 8 μL/well and centrifugation was carried out at 1000 rpm for 1 minute. The resulting mixture was incubated at 25° C. for 40 minutes. The RLU (Relative luminescence unit) signal values were read using a Biotek multimode microplate reader and the percent inhibition was calculated according to the following formula: [1−(LUM_{compound}−LUM_{positive control})/(LUM_{negative control}−LUM_{positive control})]×100. IC₅₀ values were calculated using Graphpad 7.0 software using a four-parameter non-linear fit equation. The specific results are shown in Table 1.

TABLE 1
Inhibitory activity against AAK1

| Compound No. | IC₅₀/nM |
|---|---|
| Compound 1 | 14.72 |
| Compound 2 | 10.92 |
| Compound 5 | 11.47 |
| Compound 7 | 19.82 |
| Compound 8 | 22.26 |
| Compound 9 | 37.73 |
| Compound 10 | 13.74 |

Conclusion: the compounds of the present disclosure show relatively high inhibitory activity against the AAK1 receptor.

### Example 3: Pharmacokinetic Test of Dogs

Experimental animals: male beagle dogs, about 8 to 11 kg, 6 beagle dogs/compound, purchased from Beijing Marshall Biotechnology Co., Ltd.

Experimental method: on the day of the experiment, 12 beagle dogs were randomly grouped according to their body weights. The animals were fasted with water available for 12 to 14 h one day before the administration and were fed 4 h after the administration. Administration was performed as per Table 2.

TABLE 2
Administration information

| | | Administration information | | | | | |
|---|---|---|---|---|---|---|---|
| | | | Administration | Administration | Administration | | |
| | Number | Test | dosage | concentration | volume | Collected | Mode of |
| Group | Male | compound | (mg/kg) | (mg/mL) | (mL/kg) | sample | administration |
| G1 | 3 | LX-9211 | 1 | 1 | 1 | Plasma | Intravenous |
| | | | | | | | administration |
| G2 | 3 | | 3 | 0.6 | 5 | Plasma | Intragastric |
| | | | | | | | administration |
| G3 | 3 | Compound | 1 | 1 | 1 | Plasma | Intravenous |
| | | 9 | | | | | administration |
| G4 | 3 | | 3 | 0.6 | 5 | Plasma | Intragastric |

Note:
vehicle for intravenous administration: 5% DMA + 5% Solutol + 90% Saline; vehicle for intragastric administration: 0.5% MC
(DMA: dimethylacetamide; Solutol: polyethylene glycol-15-hydroxystearate; Saline: physiological saline; MC: methylcellulose)

Before and after the administration, 1 ml of blood was taken from the jugular veins or limb veins, and placed in an EDTAK2 centrifuge tube. Centrifugation was performed at 5000 rpm at 4° C. for 10 min, and plasma was collected. Blood sampling time points for both the LX9211 intravenous administration group and intragastric administration group include: 0 min, 5 min, 15 min, 30 min, 1 h, 2 h, 4 h, 6 h, 8 h, 10 h, 12 h, and 24 h, and blood sampling time points for both the compound 9 intravenous administration group and intragastric administration group include: 0 min, 5 min, 15 min, 30 min, 1 h, 2 h, 4 h, 6 h, 8 h, 10 h, 12 h, 24 h, and 48 h. Before analysis and detection, all samples were stored at −80° C., and the samples were quantitatively analyzed by LC-MS/MS. The experimental results are shown in Table 3.

TABLE 3
Pharmacokinetic parameters of test compounds in plasma of beagle dogs

| Test | Mode of | CL | Vdₛₛ | AUC₀₋ₜ | |
|---|---|---|---|---|---|
| compound | administration | (mL/min/kg) | (L/kg) | (hr × ng/mL) | F (%) |
| LX-9211 | i.v. (1 mg/kg) | 22.2 ± 7.7 | 9.08 ± 1.4 | 751 ± 204 | — |
| | i.g. (3 mg/kg) | — | — | 500 ± 183 | 22.2 ± 8.1 |
| Compound 9 | i.v. (1 mg/kg) | 39.8 ± 10 | 23.6 ± 3.8 | 398 ± 94 | — |
| | i.g. (3 mg/kg) | | | 1353 ± 83 | 113 ± 6.9 |

—: not applicable.
Notes:
LX-9211 has a structure of

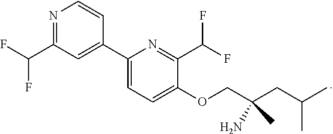 [SMILES: CC(C)C[C@](C)(N)COc1ccc(-c2ccnc(C(F)F)c2)nc1C(F)F]

Conclusion: the compounds of the present disclosure possess good pharmacokinetic characteristics.

### Example 4: Test for hERG Potassium Ion Channel

Experimental platform: electrophysiological manual patch-clamp system

Cell line: Chinese hamster ovary (CHO) cell line stably expressing hERG potassium ion channel

Experimental method: in CHO (Chinese Hamster Ovary) cells stably expressing hERG potassium channel, the whole cell patch-clamp technique was used to record hERG potassium channel current at room temperature. The glass microelectrode was made of a glass electrode blank (BF150-86-10, Sutter) by a puller. The tip resistance after filling the liquid in the electrode was about 2-5 MΩ. The glass microelectrode can be connected to the patch-clamp amplifier by inserting the glass microelectrode into an amplifier probe. The clamping voltage and data recording were controlled and recorded by the pClamp 10 software through a computer. The sampling frequency was 10 kHz, and the filtering frequency was 2 kHz. After the whole cell records were obtained, the cells were clamped at −80 mV, and the step voltage that induced the hERG potassium current (I hERG) was depolarized from −80 mV to +20 mV for 2 s, then repolarized to −50 mV, and returned to −80 mV after 1 s. This voltage stimulation was given every 10 s, and the administration process was started after the hERG potassium current was confirmed to be stable (at least 1 minute). The compound was administered for at least 1 minute at each test concentration, and at least 2 cells (n≥2) were tested at each concentration.

Data processing: data analysis processing was carried out by using pClamp 10, GraphPad Prism 5 and Excel software. The inhibition degree of hERG potassium current (peak value of hERG tail current induced at −50 mV) at different compound concentrations was calculated by the following formula:

$$Inhibition \% = [ 1 - ( I - Io ) ] \times 100 \%$$

where Inhibition % represents the percentage of inhibition of hERG potassium current by the compound, and I and Io represent the amplitude of hERG potassium current after and before the administration, respectively.

Compound IC50 was calculated using GraphPad Prism 5 software by fitting according to the following equation:

$$Y = Bottom + ( Top - Bottom ) / ( 1 + 10 \hat{} ( ( Log IC 50 - X ) \times HillSlope ) )$$

Among the equation, ×represents the Log value of the tested concentration of the test sample, Y represents the inhibition percentage at the corresponding concentration, and Bottom and Top represent the minimum and maximum inhibition percentage, respectively.

Experimental results: IC50 values of the inhibitory effect of the test compounds on hERG potassium channel current are shown in Table 4.

TABLE 4
Inhibition of test compounds on hERG potassium channel current

| | Inhibition rate at the highest | |
|---|---|---|
| Test compound | concentration tested | IC₅₀ (μM) |
| LX-9211 | 102.2 ± 4.08% at 40 μM | 1.82 |
| Compound 2 | 86.6 ± 1.77% at 40 μM | 8.75 |
| Compound 9 | 68.9 ± 1.80% at 40 μM | 24.1 |

### Example 5: Spinal Nerve Ligation (SNL)-Induced Mouse Model of Neuropathic Pain

Male C57BL/6J mice (8 weeks old) purchased from Jinan Pengyue Experimental Animal Breeding Co., Ltd. were adaptively raised for one week and then the models were established. The specific establishment method comprises the following steps:
1) disinfecting the surgical instrument and ligature;
2) anesthetizing the mice with isoflurane and then placing the mice on the operating table in the prone position;
3) shaving the fur near the hip bones of the mice and preparing the skin, and making an incision of about 2 cm along the spine;
4) isolating the fascia along the spine, performing blunt dissection of the muscles, and exposing the transverse process of L5;
5) carefully cutting the transverse process of L5 with forceps and exposing the L5 spinal nerve;
6) carefully isolating the L5 nerve with a glass dissecting needle, and ligating the L5 nerve with a 5-0 ligature; and
7) suturing the muscles and skin and disinfecting same with iodophor.

The mice that were unsuccessful in modeling were eliminated the next day after modeling (marker for successful modeling: mice with their hind paws curled up). After modeling, the mice were stroked for 3 to 5 minutes every day to ensure that the animals were familiar with the experimenter, and then the mice were placed on a metal pain measuring frame for 40 to 60 minutes of adaptation. After 3 days of acclimatization, Von Frey filaments (Aesthesio®; 0.16 g, 0.4 g, 0.6 g, 1.0 g, 1.4 g and 2.0 g) were used to test the pre-administration baseline values of the animals (Ascending testing approach). Each animal was tested twice and average values were taken, with intervals of at least 5 minutes. The animals were grouped according to the baseline values (10 animals per group). After the grouping, LX-9211 (1 and 10 mg/kg), compound 2 (1 and 10 mg/kg) or vehicle (40% PEG-400+10% ethanol+15% Tween 80+35% physiological saline) was administered intragastrically. The mice were tested for the mechanical pain threshold (MPT) at 1, 3 and 6 hours after the administration. Time-MPT curve was plotted using GraphPad 8.3.0, and statistical analysis was performed.

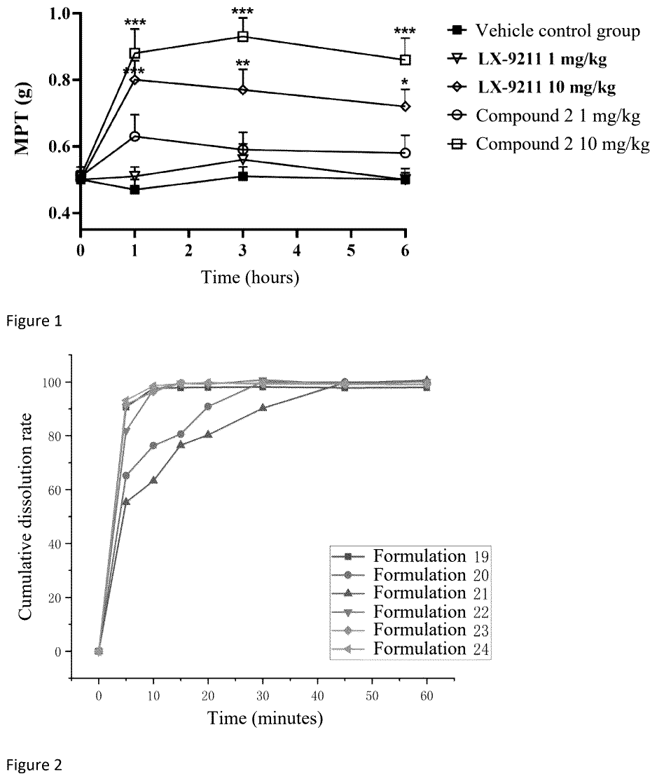

Results and conclusion: the results are shown in FIG. 1. At 1, 3 and 6 hours after a single administration, LX-9211 and compound 2 (both at the dose of 10 mg/kg) effectively increased the pain threshold of mice after SNL modeling. The analgesic efficacy of LX-9211 (10 mg/kg) reached the peak at 1 hour after the administration, and the efficacy gradually decreased thereafter. The analgesic efficacy of compound 2 (10 mg/kg) reached the peak at 3 hours after the administration and tended to be stable at 1 hour to 6 hours after the administration. Compound 2 had better efficacy than LX-9211 at 3 and 6 hours after the administration. The above data show that as an analgesic, compound 2 has better pharmacodynamic activity than LX-9211.

### Example 6: Mouse Brain/Blood Ratio Test

### 6.1 Experimental Animals

male ICR mice, 20 to 25 g, 9 mice/compound. purchased from Chengdu Ddossy Experimental Animals Co., Ltd.

### 6.2 Experimental Design

on the day of the experiment, 18 ICR mice were randomly grouped according to their body weights. The animals were fasted with water available for 12 to 14 h one day before the administration and were fed 4 h after the administration.

TABLE 5
Administration information

| | | Administration information | | | | | |
|---|---|---|---|---|---|---|---|
| | | | Administration | Administration | Administration | | |
| | Number | Test | dosage | concentration | volume | Collected | Mode of |
| Group | Male | compound | (mg/kg) | (mg/mL) | (mL/kg) | sample | administration |
| G1 | 9 | LX9211 | 10 | 1 | 10 | Plasma | Intragastric |
| | | | | | | | administration |
| G2 | 9 | Compound | 10 | 1 | 10 | Plasma | Intragastric |
| | | 2 | | | | | administration |

Note:
vehicle for intragastric administration: 40% PEG-400 + 10% Ethanol + 15% Tween 80 + 35% Saline;
(Saline; Ethanol; Tween 80)

After intragastric administration, whole blood and brain tissue were collected at 0.5 h, 4 h and 24 h, and the whole blood was centrifuged to separate the plasma. The brain tissue was rinsed with cold physiological saline to remove the residual blood on the surface, drained out and then homogenized. Before analysis and detection, all samples were stored at −80 C, and the samples were quantitatively analyzed by LC-MS/MS.

The test results are shown in Table 6.

TABLE 6
Pharmacokinetic parameters of compounds in plasma of mice

| | | Plasma | Brain tissue | Brain/ |
|---|---|---|---|---|
| Test | Mode of | AUC₀₋ₜ | AUC₀₋ₜ | plasma |
| compound | administration | (hr*ng/mL) | (hr*ng/g) | ratio |
| LX9211 | i.g. (10 mg/kg) | 6643 | 110424 | 16.6 |
| Compound 2 | i.g. (10 mg/kg) | 572 | 36853 | 64.5 |

—: not applicable.

Conclusion: the compounds of the present disclosure, especially compound 2, have a high brain penetrability.

### Example 7: Compositions of Compound 3

### 7.1 Formulations 1 to 6

TABLE 7
Formulations of AAK1 inhibitor-containing compositions (1)

| Name of | | Amount (%) | | | | | |
|---|---|---|---|---|---|---|---|
| raw | | Formulation | Formulation | Formulation | Formulation | Formulation | Formulation |
| materials | Function | 1 | 2 | 3 | 4 | 5 | 6 |
| Compound | Active | 10 | 10 | 10 | 10 | 10 | 10 |
| 3 | drug | | | | | | |
| Silica | Infiltrating | / | 2 | 5 | 10 | 10 | 10 |
| Talc | agent | / | / | / | / | 5 | 10 |
| Microcrystalline | Diluent | 60 | 58 | 55 | 50 | 45 | 40 |
| cellulose | | | | | | | |
| Lactose | | 24.5 | 24.5 | 24.5 | 24.5 | 24.5 | 24.5 |
| Low- | Disintegrant | 5 | 5 | 5 | 5 | 5 | 5 |
| substituted | | | | | | | |
| hydroxypropyl | | | | | | | |
| cellulose | | | | | | | |
| Magnesium | Lubricant | 0.5 | 0.5 | 0.5 | 0.5 | 0.5 | 0.5 |
| stearate | | | | | | | |

Tablets containing compositions of compound 3 were prepared according to the formulations in the table: first, compound 3 and an infiltrating agent were co-grinded and mixed for 15 minutes, then a diluent and a disintegrant were added and mixed in a mixer for 15 minutes, and finally a lubricant was added and mixed for 3 minutes to obtain compositions containing compound 3; the compositions were compressed into tablets using a tablet press.

We observed whether, during the process, there were phenomena such as agglomeration in the composition or the composition adhering to the equipment resulting from the softening or melting of compound 3 in the above-mentioned formulations. In addition, we investigated the content uniformity of the finished product.

TABLE 8
Experimental results

| Phenomenon | Formulation | Formulation | Formulation | Formulation | Formulation | Formulation |
|---|---|---|---|---|---|---|
| and result | 1 | 2 | 3 | 4 | 5 | 6 |
| Is there | No | No | No | No | No | No |
| agglomeration | | | | | | |
| in the | | | | | | |
| composition? | | | | | | |
| Is there | No | No | No | No | No | No |
| adhesion to | | | | | | |
| equipment? | | | | | | |
| Content | 4.3 | 3.8 | 5.2 | 4.6 | 4.3 | 5.5 |
| uniformity | | | | | | |
| (A + 2.2S) | | | | | | |

The results indicate that, regardless of whether and infiltrating agent is added to the formulation, no phenomena of agglomeration or adhesion to equipment resulting from the softening or melting of compound 3 have occurred.

### 7.2 Formulations 7 to 12

TABLE 9
Formulations of AAK1 inhibitor-containing compositions (2)

| Name of | | Amount (%) | | | | | |
|---|---|---|---|---|---|---|---|
| raw | | Formulation | Formulation | Formulation | Formulation | Formulation | Formulation |
| materials | Function | 7 | 8 | 9 | 10 | 11 | 12 |
| Compound | Active | 20 | 40 | 80 | 40 | 40 | 40 |
| 3 | drug | | | | | | |
| Silica | Infiltrating | / | / | / | 3 | 6 | 10 |
| Magnesium | agent | / | / | / | 2 | 4 | 5 |
| silicate | | | | | | | |
| Microcrystalline | Diluent | 45 | 35 | 10 | 30 | 25 | 20 |
| cellulose | | | | | | | |
| Mannitol | | 29.5 | 19.5 | 4.5 | 19.5 | 19.5 | 19.5 |
| Crospovidone | Disintegrant | 5 | 5 | 5 | 5 | 5 | 5 |
| Magnesium | Lubricant | 0.5 | 0.5 | 0.5 | 0.5 | 0.5 | 0.5 |
| stearate | | | | | | | |

Tablets containing compositions of compound 3 were prepared according to the formulations in the table: first, compound 3 and an infiltrating agent were sieved and mixed 5 times, then mixed in a mixer for 15 minutes, and then a diluent and a disintegrant were added and mixed for 15 minutes, and finally a lubricant was added and mixed for 3 minutes to obtain compositions containing compound 3; the compositions were compressed into tablets using a tablet press.

We observed whether, during the process, there were phenomena such as agglomeration in the composition or the composition adhering to the equipment resulting from the softening or melting of compound 3 in the above-mentioned formulations. In addition, we investigated the content uniformity of the finished product.

TABLE 10
Experimental results

| Phenomenon | | | | | | |
|---|---|---|---|---|---|---|
| and | Formulation | Formulation | Formulation | Formulation | Formulation | Formulation |
| result | 7 | 8 | 9 | 10 | 11 | 12 |
| Is there | / | Yes | Yes | No | No | No |
| agglomeration | | | | | | |
| in the | | | | | | |
| composition? | | | | | | |
| Is there | / | Yes | Yes | / | No | No |
| adhesion to | | | | | | |
| equipment? | | | | | | |
| Content | / | 10.5 | 13.2 | 3.6 | 4.6 | 3.5 |
| uniformity | | | | | | |
| (A + 2.2S) | | | | | | |

Conclusion: the addition of an appropriate amount of an infiltrating agent to the formulation can significantly mitigate the softening/melting phenomenon of compound 3 during the preparation process, and as a result, the tablets exhibit better content uniformity.

### 7.3 Formulations 13 to 18

TABLE 11
Formulations of AAK1 inhibitor-containing compositions (3)

| Name of | | Amount (%) | | | | | |
|---|---|---|---|---|---|---|---|
| raw | | Formulation | Formulation | Formulation | Formulation | Formulation | Formulation |
| materials | Function | 13 | 14 | 15 | 16 | 17 | 18 |
| Compound | Active | 20 | 40 | 80 | 40 | 40 | 40 |
| 3 | drug | | | | | | |
| Silica | Infiltrating | / | / | / | 5 | 10 | 15 |
| | agent | | | | | | |
| Pregelatinized | Diluent | 40 | 30 | 10 | 30 | 25 | 20 |
| starch | | | | | | | |
| Lactose | | 35 | 25 | 5 | 20 | 20 | 20 |
| Sodium | Disintegrant | 3 | 3 | 3 | 3 | 3 | 3 |
| carboxymethyl | | | | | | | |
| starch | | | | | | | |
| Talc | Lubricant | 2 | 2 | 2 | 2 | 2 | 2 |

Preparation method: compound 3 and an infiltrating agent were mixed in a mixer for 15 minutes, and then a diluent and a disintegrant were added and mixed for 15 minutes, and finally magnesium stearate was added and mixed for 3 minutes to obtain compositions containing compound 3; the compositions containing compound 3 were filled into capsules using a capsule filling machine.

We observed whether, during the process, there were phenomena such as agglomeration in the composition or the composition adhering to the equipment resulting from the softening or melting of compound 3 in the above-mentioned formulations. In addition, we investigated the content uniformity of the finished product.

TABLE 12
Experimental results

| Phenomenon | | | | | | |
|---|---|---|---|---|---|---|
| and | Formulation | Formulation | Formulation | Formulation | Formulation | Formulation |
| result | 13 | 14 | 15 | 16 | 17 | 18 |
| Is there | No | Yes | Yes | No | No | No |
| agglomeration | | | | | | |
| in the | | | | | | |
| composition? | | | | | | |
| Is there | No | Yes | Yes | No | No | No |
| adhesion to | | | | | | |
| equipment? | | | | | | |
| Content | 4.6 | 8.3 | 11.0 | 4.1 | 5.5 | 3.9 |
| uniformity | | | | | | |
| (A + 2.2S) | | | | | | |

Conclusion: the addition of an infiltrating agent can significantly inhibit the softening/melting phenomenon of compound 3 during the preparation process.

### 7.4 Formulations 19 to 24

TABLE 13
Formulations of AAK1 inhibitor-containing compositions (4)

| Name of | | Amount (%) | | | | | |
|---|---|---|---|---|---|---|---|
| raw | | Formulation | Formulation | Formulation | Formulation | Formulation | Formulation |
| materials | Function | 19 | 20 | 21 | 22 | 23 | 24 |
| Compound | Active | 20 | 40 | 80 | 40 | 40 | 40 |
| 3 | drug | | | | | | |
| Silica | Infiltrating | / | / | / | 5 | 10 | 15 |
| | agent | | | | | | |
| Microcrystalline | Diluent | 45 | 35 | 10 | 30 | 25 | 20 |
| cellulose | | | | | | | |
| Lactose | | 29.5 | 19.5 | 4.5 | 19.5 | 19.5 | 19.5 |
| Croscarmellose | Disintegrant | 5 | 5 | 5 | 5 | 5 | 5 |
| sodium | | | | | | | |
| Magnesium | Lubricant | 0.5 | 0.5 | 0.5 | 0.5 | 0.5 | 0.5 |
| stearate | | | | | | | |

Tablets containing compositions of compound 3 were prepared according to the formulations in the table: first, compound 3 and an infiltrating agent were sieved and mixed 5 times, then mixed in a mixer for 15 minutes, and then a diluent and a disintegrant were added and mixed for 15 minutes, and subsequently the premix was fed into a roller compactor and compacted at 0.6 kN/cm; the resultant ribbon-like material was passed through a 0.8 mm vibration mill; the milled particles were then mixed with a lubricant for 3 minutes, and finally the mixture was compressed into tablets using a tablet press.

We investigated the dissolution behavior of the tablets prepared according to the above-mentioned formulations and processes in a pH 1.0 medium, as well as the changes in their related substances when stored at 40° C.±2 C, 75% RH±5% RH for 6 months, see Table 14.

As can be seen from FIG. 2, for formulations 19 to 21, with the increase in the proportion of compound 3, the agglomeration of AAK1 resulting from its softening/melting further decreases its dissolution rate. For formulations 22 to 24, the addition of an infiltrating agent to the formulations can avoid the agglomeration resulting from the softening/melting of compound 3, thereby improving its dissolution behavior.

TABLE 14
Stability results

| | Total impurity (%) | | | | | |
|---|---|---|---|---|---|---|
| Storage | Formulation | Formulation | Formulation | Formulation | Formulation | Formulation |
| time | 19 | 20 | 21 | 22 | 23 | 24 |
| 0 days | 0.584 | 0.562 | 0.581 | 0.576 | 0.543 | 0.572 |
| 1 | 0.607 | 0.557 | 0.605 | 0.586 | 0.552 | 0.565 |
| month | | | | | | |
| 2 | 0.575 | 0.586 | 0.615 | 0.602 | 0.548 | 0.573 |
| months | | | | | | |
| 3 | 0.600 | 0.598 | 0.624 | 0.615 | 0.566 | 0.598 |
| months | | | | | | |
| 6 | 0.621 | 0.625 | 0.648 | 0.636 | 0.594 | 0.615 |
| months | | | | | | |

As can be seen from the table, regardless of whether the composition contains an infiltrating agent, it demonstrates good stability.

### 7.5 Formulations 25 to 28

TABLE 15
Formulations of AAK1 inhibitor-containing compositions (5)

| Name of raw | | Amount (mg) |
|---|---|---|
| materials | Function | Formulation 25 |
| Compound 3 | Active drug | 10 |
| Microcrystalline cellulose | Diluent | 62 |
| Lactose | | 22 |
| Croscarmellose sodium | Disintegrant | 5 |
| Magnesium stearate | Lubricant | 1 |

Preparation method: compound 3 was mixed with lactose, microcrystalline cellulose and croscarmellose sodium in a mixer for 15 minutes, and then magnesium stearate was added and mixed for 3 minutes; the mixture was then compressed into tablets using a tablet press, with the tablet hardness controlled within the range of 50 to 100 N.

TABLE 16
Formulations of AAK1 inhibitor-containing compositions (6)

| Name of raw | | Amount (mg) |
|---|---|---|
| materials | Function | Formulation 26 |
| Compound 3 | Active drug | 25 |
| Microcrystalline | Diluent | 40 |
| cellulose | | |
| Mannitol | | 30 |
| Low-substituted | Disintegrant | 3 |
| carboxymethyl | | |
| cellulose sodium | | |
| Talc | Lubricant | 2 |

Preparation method: compound 3 was mixed with mannitol, microcrystalline cellulose and low-substituted carboxymethyl cellulose sodium in a premixer for 15 minutes, and then the premix was fed into a roller compactor and compacted at 0.6 kN/cm; the resultant ribbon-like material was passed through a 0.8 mm vibration mill; the milled particles were then mixed with talc for 3 minutes, and finally the mixture was compressed into tablets using a tablet press, with the tablet hardness controlled within the range of 50 to 100 N.

TABLE 17
Formulations of AAK1 inhibitor-containing compositions (7)

| Name of raw | | Amount (mg) |
|---|---|---|
| materials | Function | Formulation 27 |
| Compound 3 | Active drug | 50 |
| Pregelatinized starch | Diluent | 75 |
| Dextrin | | 70 |
| Sodium | Disintegrant | 3 |
| carboxymethyl starch | | |
| Magnesium stearate | Lubricant | 2 |

Preparation method: compound 3 was mixed with pregelatinized starch, sodium carboxymethyl starch and dextrin in a mixer for 15 minutes, and then magnesium stearate was added and mixed for 3 minutes; the mixture was then compressed into tablets using a tablet press, with the tablet hardness controlled within the range of 50 to 100 N.

TABLE 18
Formulations of AAK1 inhibitor-containing compositions (8)

| Name of raw | | Amount (mg) |
|---|---|---|
| materials | Function | Formulation 28 |
| Compound 3 | Active drug | 100 |
| Calcium phosphate | Diluent | 80 |
| Sucrose | | 115 |
| Crospovidone | Disintegrant | 3 |
| Talc | Lubricant | 2 |

Preparation method: compound 3 was mixed with calcium phosphate, sucrose, crospovidone and talc in a mixer for 15 minutes, and then the mixture was filled into capsules using a capsule filling machine.

In accordance with Method 2 (Paddle Method) in the Dissolution and Release Test (section 0931) of the 2015 edition of the Chinese Pharmacopoeia, the dissolution curves of the tablets prepared according to formulation 25 in water, a pH 1.0 medium, a pH 4.5 medium, and a pH 6.8 medium were investigated. The rotation speed was set to 50 rpm, and the sampling time points were 5 min, 10 min, 15 min, 20 min, 30 min, 45 min, and 60 min.

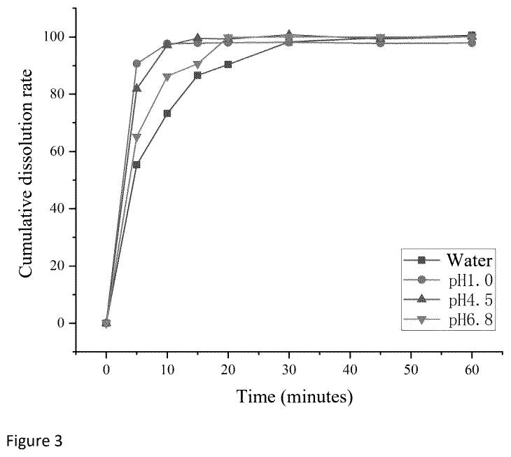

As can be seen from the results in FIG. 3, when pH=1.0, the dissolution rate of the tablets prepared according to formulation 25 can reach 90% within 5 minutes, indicating rapid dissolution; at 20 minutes, the dissolution rate of the tablets prepared according to formulation 25 in water, a pH 1.0 medium, a pH 4.5 medium, and a pH 6.8 medium can all reach 85%.

### 7.6 Study Methods and Results of Formulation Stability

The formulations prepared according to formulation 25, formulation 26, formulation 27 and formulation 28 were internally packaged in the form of aluminum-plastic blister packages, stored at 40° C.±2° C., 75% RH±5% RH, and then investigated for the changes in their related substances.

TABLE 19
Stability investigation results

| | Total impurity (%) | | | |
|---|---|---|---|---|
| | Formulation | Formulation | Formulation | Formulation |
| Storage time | 25 | 26 | 27 | 28 |
| 0 days | 0.584 | 0.562 | 0.581 | 0.576 |
| 1 month | 0.607 | 0.557 | 0.605 | 0.586 |
| 2 months | 0.575 | 0.586 | 0.615 | 0.602 |
| 3 months | 0.600 | 0.598 | 0.624 | 0.615 |
| 6 months | 0.621 | 0.625 | 0.648 | 0.636 |

As can be seen from the results in Table 19, the formulations prepared according to formulations 25-28 all exhibit relatively high stability, and their stabilities are essentially equivalent.

The pharmaceutical compositions of compound 2 were prepared by referencing formulations 1-6, 10-13 and 16-28 and processes of the pharmaceutical compositions of compound 3. No phenomena of agglomeration or adhesion to equipment were observed. The content uniformity (A+2.2S) ranged from 3 to 5.5, and all the pharmaceutical compositions demonstrated relatively high stability.

1. A pharmaceutical composition, comprising:
an active ingredient, wherein the active ingredient is selected from a compound of formula (I), or a stereoisomer or a pharmaceutically acceptable salt thereof: 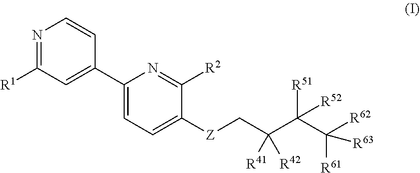
wherein
Z is selected from NH or O;
R¹ and R² are each independently selected from H, deuterium, halogen, amino, —COOH, cyano, sulfonyl, aminoacyl, C₁₋₆ alkyl, halo C₁₋₆ alkyl, or deuterated C₁₋₆ alkyl, wherein the alkyl is optionally further substituted with 1-3 R^{A} substituents;
R⁴¹ and R⁴² are each independently selected from H, deuterium, amino, C₁₋₆ alkyl, halogen, cyano, hydroxyl, halo C₁₋₆ alkyl, or deuterated C₁₋₆ alkyl;
R⁵¹ and R⁵² are each independently selected from H, deuterium, amino, or halogen;
R⁶¹, R⁶² and R⁶³ are each independently selected from H, deuterium, halogen, amino, cyano, hydroxyl, C₁₋₆ alkyl, halo C₁₋₆ alkyl, or deuterated C₁₋₆ alkyl;
or, R⁵¹ and R⁶¹, or R⁶¹ and R⁶² together with the carbon atom(s) to which they are each attached form a double bond;
R^{A} is selected from deuterium, halogen, amino, cyano, hydroxyl, C₁₋₆ alkyl, halo C₁₋₆ alkyl, deuterated C₁₋₆ alkyl, C₁₋₆ alkoxy, halo C₁₋₆ alkoxy, deuterated C₁₋₆ alkoxy, or hydroxy C₁₋₆ alkyl,
provided that when Z is selected from O, 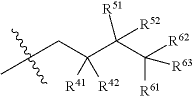
does not form the following structures: 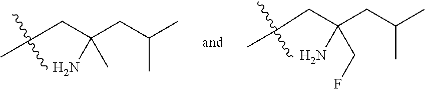
a non-active ingredient;
wherein the active ingredient is present in the pharmaceutical composition in an amount of 5% to 90% w/w.

2. The pharmaceutical composition according to claim 1, wherein
Z is O;
R1 and R2 are selected from halo C1-2 alkyl;
R51 and R52 are each independently selected from H or deuterium;
R41 and R42 are each independently selected from amino or C1-2 alkyl;
R61, R62 and R63 are each independently selected from H, deuterium, or C1-2 alkyl.

3. The pharmaceutical composition according to claim 1, wherein the compound of formula (I) is selected from one of the following structures:

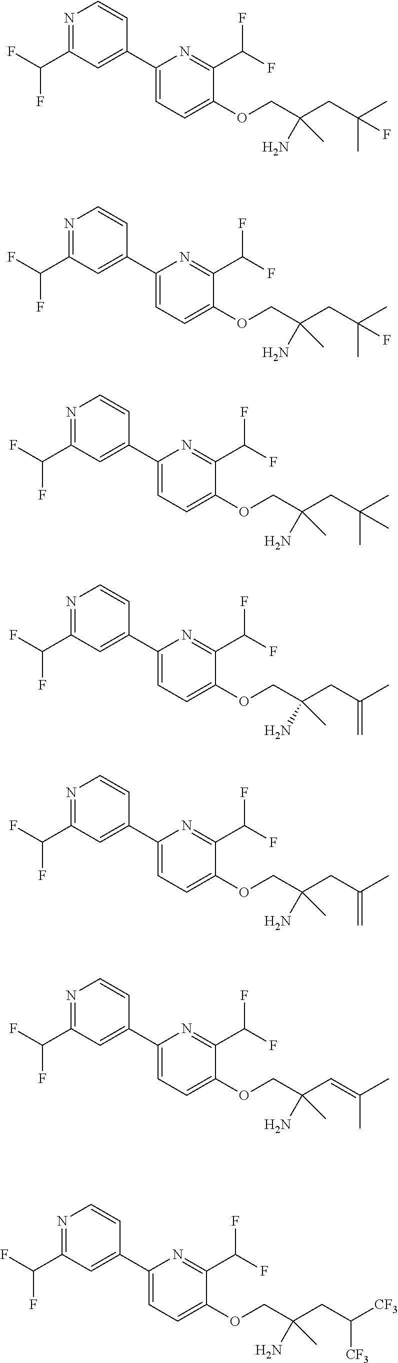

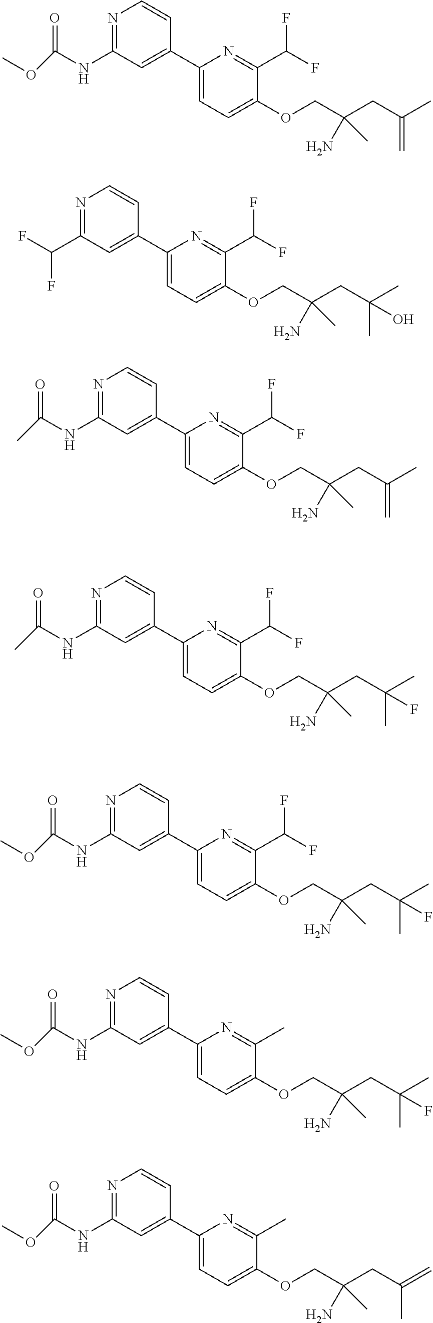

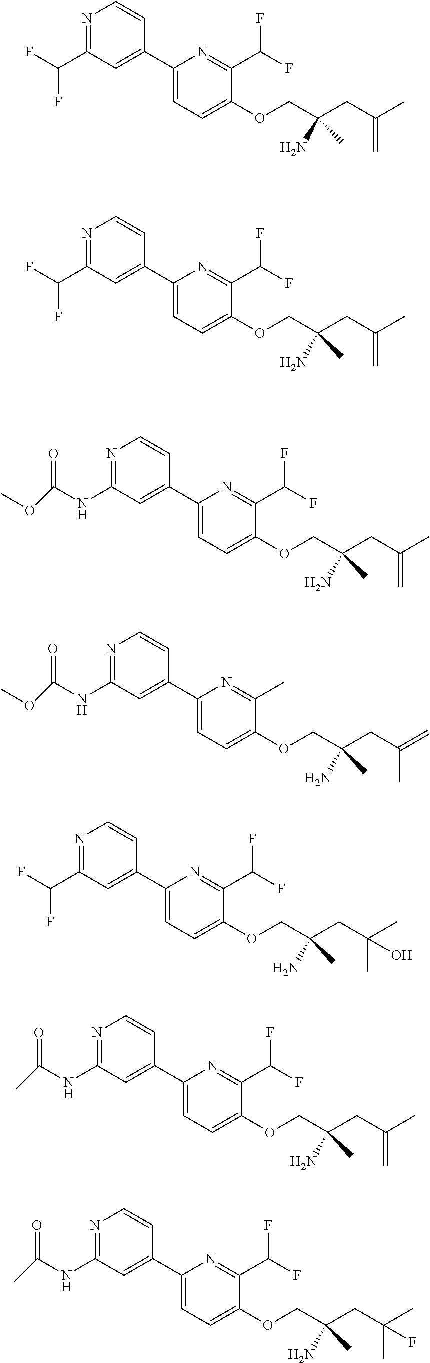

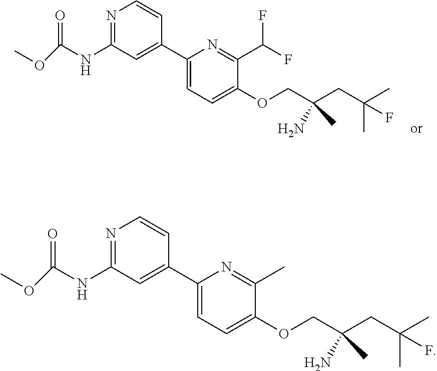 [SMILES: COC(=O)Nc1cc(-c2ccc(OC[C@@](C)(N)CC(C)(C)F)c(C(F)F)n2)ccn1 ; COC(=O)Nc1cc(-c2ccc(OC[C@@](C)(N)CC(C)(C)F)c(C)n2)ccn1]

4. The pharmaceutical composition according to claim 1, wherein the non-active ingredient is selected from a diluent.

5. The pharmaceutical composition according to claim 4, wherein the diluent is selected from one or more of starch, pregelatinized starch, dextrin, lactose monohydrate, anhydrous lactose, sucrose, microcrystalline cellulose, inorganic salts, and sugar alcohols.

6. The pharmaceutical composition according to claim 4, wherein the weight ratio of the active ingredient to the diluent is 1:0.05 to 1:10.

7. The pharmaceutical composition according to claim 4, wherein the non-active ingredient further contains one or more of an infiltrating agent, a disintegrant, and a lubricant;
the infiltrating agent is selected from one or more of silicates;
the disintegrant is selected from one or more of croscarmellose sodium, crospovidone, starch and a derivative thereof, low-substituted hydroxypropyl cellulose, low-substituted sodium hydroxymethyl cellulose, a surfactant, alginic acid and sodium alginate, and clays; and
the lubricant is selected from one or more of talc, stearic acid, metal stearate, stearate, glyceryl behenate, sodium lauryl sulfate or colloidal silica.

8. The pharmaceutical composition according to claim 7, wherein the weight ratio of the active ingredient to the infiltrating agent is 1:0.05 to 1:5;
the weight ratio of the active ingredient to the disintegrant is 1:0.01 to 1:3; and
the weight ratio of the active ingredient to the lubricant is 1:0.001 to 1:2.

9. A pharmaceutical formulation, comprising the pharmaceutical composition according to claim 1.

10. The pharmaceutical formulation according to claim 9, wherein the amount of the active ingredient in a unit formulation is from 1 mg to 100 mg.

11. The pharmaceutical formulation according to claim 9, wherein the formulation form of the pharmaceutical formulation is selected from a tablet, a granule, a capsule, or a soft capsule.

12. A method for treating an AAK1-induced related disease by inhibition or degradation of AAK1 comprising administering the pharmaceutical composition according to claim 1 to a subject.

13. The method according to claim 12, wherein the disease is selected from inflammatory pain, postoperative pain, trigeminal neuralgia, acute postherpetic neuralgia and postherpetic neuralgia, diabetic peripheral neuralgia, causalgia, occipital neuralgia, fibromyalgia, phantom limb pain, burn pain and other forms of neuralgia, neuropathy and spontaneous pain syndrome.
